(19) United States
(12) Patent Application Publication
(10) Pub. No.: US 2026/0283693 A1
(43) Pub. Date: Sep. 24, 2026

(54) URETEROSCOPE WITH INTEGRATED FLUID CHANNEL

(71) Applicant: Ventaris Surgical, Inc., San Carlos, CA (US)

(72) Inventors: Surag Mantri, East Palo Alto, CA (US); Peter Bentley, Reno, NV (US); Scott Taylor, San Martin, CA (US); Michael Walker, San Mateo, CA (US)

(21) Appl. No.: 19/570,033

(22) Filed: Mar. 17, 2026

Related U.S. Application Data
(60) Provisional application No. 63/774,632, filed on Mar. 19, 2025.

(51) Int. Cl. A61B 18/26; A61B 1/00; A61B 1/005; A61B 1/018; A61B 1/05; A61B 1/307; A61B 18/00; A61B 18/20

(52) U.S. Cl. CPC A61B 18/26; A61B 1/000094; A61B 1/0008; A61B 1/00119; A61B 1/00133; A61B 1/005; A61B 1/018; A61B 1/05; A61B 1/307; A61B 18/201; A61B 2018/00196; A61B 2018/00511; A61B 2018/00577; A61B 2018/00982; A61B 2218/002; A61B 2218/007

(57) ABSTRACT

A system includes an elongated tubular body having supply lumen and an evacuation lumen, and a distal tip having a working channel extension and a choke. The supply lumen is configured to redirect a first fluid flow from the fluid source. The evacuation lumen has a first cross-sectional area and is configured to receive the first fluid flow from the supply lumen after the first fluid flow is redirected into the evacuation lumen. The distal tip extends from a distal end of the elongated tubular body. The working channel extension is in fluid communication with the evacuation lumen and defines an aspiration opening configured to receive a second fluid flow as a result of the first fluid flow entering the evacuation lumen. The choke has a second cross-sectional area disposed within the working channel extension, wherein the second cross-sectional area is smaller than the first cross-sectional area.

### INCORPORATION BY REFERENCE TO ANY PRIORITY APPLICATIONS

This application claims priority to U.S. Provisional Patent Application No. 63/774632, filed on Mar. 19, 2025, the disclosure of which is incorporated by reference in its entirety.

### TECHNICAL FIELD

The present disclosure generally relates to medical systems and related methods, such as minimally invasive surgical devices and systems for manipulating and removing biological objects from an anatomical structure.

### DESCRIPTION OF THE RELATED ART

Minimally invasive surgeries offer numerous advantages over traditional open surgical techniques. These encompass reduced surgical trauma, decreased recovery time, shorter hospital stays, and/or potentially a diminished risk of infection and other complications. However, certain challenges persist in minimally invasive surgery, notably the requirement for precise manipulation and removal of objects within the body while preventing damage to the adjacent tissues. This precision is of paramount importance in fluid-filled environments and/or intact organs, such as the kidney, gall bladder, urinary bladder, urinary tract, blood vessels, or other body cavities or organs.

For kidney stone treatment, the two primary approaches include ureteroscopic lithotripsy and percutaneous nephrolithotomy (PCNL), which incorporates its variant, the mini-PCNL. Both procedures, whether utilizing a ureteroscope or a nephroscope, generally encounter parallel challenges, including extended operation times, an obscured visual field, difficulties in extracting residual fragments, stone retropulsion, high intra-luminal pressure, challenges with temperature regulation, and/or instrument size that limit ureteral access. Accordingly, improved systems and methods are needed for safe and effective removal of biological objects.

### SUMMARY

Described herein are systems, devices and methods for removal of a biological object from an anatomical structure. Certain systems, devices and methods may be applied to removing a biological object such as a kidney stone from a urinary tract. Certain systems and methods described herein can use a Venturi-effect or Entrainment to attract the biological object towards one or more openings of a ureteroscope.

In one or more aspects of the systems and methods disclosed herein, a system for removing a biological object from an anatomical structure is described. The system includes an elongated tubular body, a supply lumen configured to provide a fluid flow into the elongated tubular body, an evacuation lumen configured to evacuate the fluid flow and the biological object from the anatomical structure, and a distal tip positioned at the distal end of the elongated tubular body. The elongated tubular body includes a proximal end and a distal end configured to be inserted into the anatomical structure, the elongated tubular body further including one or more aspiration openings configured to receive the biological object. The distal tip includes an optically transparent material.

In some examples, the distal tip further includes a cup, a choke, and a working channel extension. In some examples, the cup includes an annular wall extending distally away from the distal end of the elongated tubular body. In some examples, the cup is formed of a polymer. In some examples, the cup is formed of a jewel. In some examples, the cup has a length between about 0.1 mm and about 2.0 mm. In some examples, the choke includes a constricted lumen. In some examples, the choke is configured to be thermal resistant and laser resistant. In some examples, the choke is formed of a jewel. In some examples, the choke has a length of about 0.1 mm to about 1.0 mm. In some examples, the supply lumen is configured to provide the fluid flow into the elongated tubular body as a liquid jet. In some examples, the supply lumen is configured to induce a Venturi-assisted suction at the one or more aspiration openings. In some examples, the supply lumen is configured to induce an Entrainment-assisted suction at the one or more aspiration openings. In some examples, the supply lumen extends distally along the elongated tubular body and includes a bend configured to redirect the fluid flow in a proximal direction. In some examples, the supply lumen includes a J-bend. In some examples, the supply lumen includes an incidence angle between about 10 degrees and about 60 degrees. In some examples, the supply lumen is positioned at a radial distance from an inner surface of the evacuation lumen. In some examples, the radial distance is between about 0.0 mm and about 0.5 mm. In some examples, the elongated tubular body further includes one or more light sources and an image sensor. In some examples, the one or more aspiration openings are positioned at a distal face of the elongated tubular body. In some examples, the elongated tubular body further includes one or more vent openings. In some examples, the one or more vent openings are offset from the distal end of the elongated tubular body by a distance of about 2.0 mm to about 10.0 mm. In some examples, the one or more vent openings are circular. In some examples, the one or more vent openings are longitudinal slots. In some examples, the one or more vent openings are slots arranged orthogonally to the longitudinal axis of the elongated tubular body. In some examples, the choke is clover shaped. In some examples, the choke is circular shaped. In some examples, the choke includes a plurality of slots. In some examples, the elongated tubular body is configured to receive an ablation device. In some examples, the ablation device is an ultrasonic device. In some examples, the ablation device is a laser device. In some examples, the system further includes a secondary lumen. In some examples, the system includes a ureteroscope configured to be inserted into a urinary tract and the biological object includes a kidney stone. In some examples, the system further comprises a reciprocation mechanism. In some examples, the reciprocation mechanism is configured to move the ablation device longitudinally. In some examples, the reciprocation mechanism automated is configured to move the ablation device between about 0.1 mm and about 2.0 mm.

In some aspects of the systems and methods disclosed herein, a system for removing a biological object from a location internal to a subject is described. The system includes an elongated tubular body and a distal tip extending distally from a distal end of the elongated tubular body. The elongated tubular body includes a supply lumen and an evacuation lumen. The supply lumen extends distally from a proximal end of the elongated tubular body, wherein the supply lumen is configured to be in fluid communication with a fluid source and configured to redirect a first fluid flow from the fluid source. The evacuation lumen has a first cross-sectional area extending from a distal end of the elongated tubular body to the proximal end of the elongated tubular body, wherein the evacuation lumen is configured to receive the first fluid flow from the supply lumen after being redirected into the evacuation lumen. The distal tip includes a working channel extension and a choke. The working channel extension is in fluid communication with the evacuation lumen, wherein a distal end of the working channel extension defines an aspiration opening configured to receive a second fluid flow as a result of the first fluid flow entering the evacuation lumen. The choke has a second cross-sectional area disposed within the working channel extension, wherein the second cross-sectional area is smaller than the first cross-sectional area.

In some examples, the choke is selected from the group consisting of a three clover shape, a four clover shape, a plurality of slots, a plurality of aspiration openings, and two hemispheric aspiration openings. In some examples, the distal tip further includes a cup, and wherein: the choke has an axial length between about 0.1 mm to about 0.5 mm; and the cup has an axial length between about 0.1 mm and about 2.0 mm. In some examples, the distal tip is formed from a jewel. In some examples, the system further includes an ablation device extending through the evacuation lumen and the distal tip. In some examples, the evacuation lumen is configured to receive an ablation device, and wherein the system further includes a reciprocating mechanism configured to reciprocate the ablation device in the evacuation lumen. In some examples, the choke supports the ablation device.

In some aspects of the systems and methods disclosed herein, a system for removing a biological object from a location internal to a subject is described. The system includes an elongated tubular structure and a distal tip extending distally from a distal end of the elongated tubular structure. The elongated tubular structure includes a working channel having a first effective cross-sectional area. The distal tip includes a working channel extension and a choke. The working channel extension extends from a distal end of the distal tip to a proximal end of the distal tip, wherein the working channel extension is in fluid communication with the working channel. The choke positioned within the working channel extension at an axial location between a distal end of the working channel extension and a proximal end of the working channel extension. The choke has a second effective cross-sectional area smaller than the first effective cross-sectional area. The system is configured to aspirate the biological object through the choke, the working channel extension, and the working channel.

In some examples, the choke is clover shaped having four fingers extending radially inward from the distal tip. In some examples, the choke is clover shaped having three fingers extending radially inward from the distal tip. In some examples, the choke includes a plurality of slots. In some examples, the choke includes a plurality of aspiration openings. In some examples, the choke includes two hemispheric openings. In some examples, the elongated tubular structure further includes an optical sensor positioned along a first axis parallel to a central axis of the working channel and a distal end of the working channel extension is axially displaced from a distal end of the optical sensor by a distance. In some examples, the system further includes an ablation device extending through the working channel and the working channel extension, wherein a distal end of the ablation device is axially aligned with the distal end of the working channel extension. In some examples, the distance is a first distance, wherein the distal end of the distal tip is axially displaced from the optical sensor by a second distance. In some examples, the first distance maintains a separation between a distal end of an ablation device and the optical sensor and the second distance maintains a separation between an aspiration opening and the optical sensor. In some examples, the distal tip further includes a cup, wherein the cup extends distally from a distal end of the working channel extension.

In some aspects of the systems and methods disclosed herein, a system for removing a biological object from a location internal to a subject is described. The system includes an elongated tubular body, a distal tip extending distally from a distal end of the elongated tubular body, an ablation device, and a controller. The elongated tubular body includes an optical sensor, a supply lumen configured to be in fluid communication with a fluid source, and an evacuation lumen in fluid communication with the supply lumen. The distal tip includes a working channel extension and a choke. The working channel extension is in fluid communication with the evacuation lumen, wherein a distal end of the working channel extension defines an aspiration opening configured to be in fluid communication with the location internal to the subject. The choke is disposed within the working channel extension. The ablation device extends through the evacuation lumen and the distal tip. The controller is configured to receive optical signals from the optical sensor and differentiate between tissue and the biological object.

In some examples, the supply lumen introduces a liquid jet with fluid from the fluid source into the evacuation lumen to create a vacuum within the working channel extension, wherein the controller is configured to automatically control a level of the vacuum based at least partially on a signal from a flow sensor. In some examples, the controller is configured to automatically control the ablation device based at least partially on a differentiation between a tissue and the biological object. In some examples, the system further includes a reciprocating mechanism coupled to the ablation device, wherein the controller is configured to automatically control, based at least partially on the optical signals, the reciprocating mechanism to cause movement of the ablation device relative to the elongated tubular body.

In some aspects of the systems and methods disclosed herein, a system for facilitating removal of a biological object from a location internal to a subject is described. The system includes an elongated tubular structure and a distal tip projecting from a distal end of the elongated tubular structure. The elongated tubular structure includes a working channel having a first effective cross-sectional area and an optical sensor positioned adjacent to the working channel. The distal tip includes a working channel extension and a restriction. The working channel extension is defined through the distal tip and in fluid communication with the working channel. The restriction is defined within the working channel extension and having a second effective cross-sectional area which is less than the first effective cross-sectional area. The optical sensor is positioned proximally of a distal end of the working channel extension by a first distance and is further positioned proximally of the restriction by a second distance which is less than the first distance.

In some examples, the restriction is clover shaped having four fingers extending radially inward from the distal tip. In some examples, the restriction is clover shaped having three fingers extending radially inward from the distal tip. In some examples, the restriction includes a plurality of slots. In some examples, I the restriction includes a plurality of aspiration openings. In some examples, the restriction includes two hemispheric openings. In some examples, the optical sensor is positioned along a first axis parallel to a central axis of the working channel and the distal end of the working channel extension is axially displaced from a distal end of the optical sensor. In some examples, the system further includes an ablation device extending through the working channel and the working channel extension, wherein a distal end of the ablation device is axially aligned with the distal end of the working channel extension. In some examples, the distal end of the distal tip is axially displaced from the optical sensor by the second distance. In some examples, the first distance maintains a separation between a distal end of an ablation device and the optical sensor and the second distance maintains a separation between an aspiration opening and the optical sensor. In some examples, the distal tip further includes a cup, wherein the cup extends distally from the distal end of the working channel extension.

In some aspects of the systems and methods disclosed herein, a system for removing a biological object from a location internal to a subject is described. The system includes a supply lumen, an evacuation lumen, an aspiration opening, and a distal tip extending distally from a distal end of the evacuation lumen. The supply lumen is configured to receive a first fluid flow and to redirect the first fluid flow from a first direction to a second direction. The evacuation lumen is configured to receive the first fluid flow and a second fluid flow travelling in the second direction from the location internal to the subject toward the evacuation lumen. The second fluid flow being configured to carry at least a portion of the biological object from the location internal to the subject toward the evacuation lumen. The aspiration opening is in fluid communication with the evacuation lumen, wherein the aspiration opening is configured to receive the second fluid flow from the location internal to the subject as a result of the first fluid flow entering the evacuation lumen. The distal tip includes a working channel extension extending from a distal end of the distal tip to a proximal end of the distal tip, wherein the working channel extension is in fluid communication with the evacuation lumen. The distal tip is configured to enhance visibility for an optical sensor configured to provide images of the location internal to the subject. The distal tip is further configured to prevent the biological object from clogging the evacuation lumen.

In some examples, the distal tip includes a choke positioned in the working channel extension and defining one or more constrictions at one or more axial positions between the distal end and the proximal end of the distal tip, and wherein the choke has an effective cross-sectional area smaller than a cross-sectional area of the evacuation lumen. In some examples, the distal tip further includes a cup having an annular wall extending distally from the distal end of the evacuation lumen, and wherein the cup is configured to retain the biological object near the aspiration opening. In some examples, the distal tip is formed from a laser energy resistant material. In some examples, the distal tip is formed from an optically transparent material. In some examples, the evacuation lumen and the distal tip are configured to receive an ablation device. In some examples, the system further includes a mechanism configured to move the ablation device in the evacuation lumen.

In one or more aspects of the systems and methods disclosed herein, a method is described. The method can be a method of operating the system described above.

In one or more aspects of the systems and methods disclosed herein, a system, device, and/or method is described. The system, device, and/or method is as illustrated and/or described herein.

### BRIEF DESCRIPTION OF THE DRAWINGS

Non-limiting examples of the present disclosure will be described by way of example with reference to the accompanying figures. For purposes of clarity, not every component is labeled in every figure, nor is every component of each example of the disclosure shown where illustration is not necessary to allow those of ordinary skill in the art to understand the disclosure.

FIG. 1 is a frontal, partial cross-sectional view of a patient prepared for a ureteroscopy.

FIG. 2A is a frontal, partial cross-sectional view of a ureteroscope being inserted into a patient.

FIG. 2B is a frontal, partial cross-sectional view of a ureteroscope being positioned within a patient's kidney.

FIGS. 2C-2H are frontal, partial cross-sectional views of steps of removing a kidney stone from a patient's kidney.

FIG. 3A is a perspective view of a distal end of a ureteroscope.

FIG. 3B is a cross-sectional perspective view of the distal end of the ureteroscope of FIG. 3A.

FIGS. 3C-3D are cross-sectional views of the distal tip of the ureteroscope of FIG. 3A showing a fluid flow path.

FIG. 3E is a front view of the distal end of the ureteroscope of FIG. 3A showing a fluid flow path.

FIG. 3F is a cross-sectional top view of the distal tip of the ureteroscope of FIG. 3A showing a top view of the fluid flow path of FIG. 3C.

FIG. 3G is a cross-sectional perspective view of the distal tip of the ureteroscope of FIG. 3A showing relative dimensions.

FIG. 4A is a perspective view of an example of a vent opening of a ureteroscope.

FIG. 4B is a perspective view of another example of a vent opening of a ureteroscope.

FIG. 4C is a perspective view of another example of a vent opening of a ureteroscope.

FIG. 5A is a perspective view of an example of a distal tip of a ureteroscope with a four clover-shaped opening.

FIG. 5B is a perspective view of the example of the distal tip of FIG. 5A with an ablation device.

FIG. 6A is a perspective view of another example of a distal tip of a ureteroscope with a dual slot opening.

FIG. 6B is a cross-sectional side view of the distal tip of FIG. 6A showing a volume between the proximal side of the dual slot opening and a supply lumen.

FIG. 7 is a perspective view of an example of a distal tip of a ureteroscope with a plurality of openings.

FIG. 8 is a perspective view of an example of a distal tip of a ureteroscope with two hemispherical openings.

FIG. 9 is a perspective view of an example of a distal tip of a ureteroscope with a three clover-shaped opening.

FIGS. 10A-10B are perspective views of an example of a distal tip of a ureteroscope without a cup.

FIG. 11 is a perspective view of an example of a distal tip of a ureteroscope without a cup.

FIG. 12 is a bottom perspective view of a distal end of a ureteroscope with two supply lumens.

FIG. 13A is a perspective view of a distal end of a ureteroscope with a secondary lumen extending along an outer surface of the ureteroscope.

FIG. 13B is a perspective view of a distal end of a ureteroscope with an integrated outer tube surrounding the ureteroscope.

FIG. 13C is a perspective view of a distal end of a ureteroscope with an integrated secondary lumen.

FIG. 14A is an example of a field of view and angle of view of a ureteroscope.

FIG. 14B is another example of a field of view and angle of view of a ureteroscope.

FIG. 14C is another example of a field of view and angle of view of a ureteroscope.

FIGS. 15A-15C are side views of a helical supply lumen extending around a ureteroscope.

FIG. 15D is a side view of a zigzag/sinusoidal supply lumen extending along a ureteroscope.

FIG. 16 is a hardware block diagram of a ureteroscope.

FIG. 17 is a fluid flow block diagram of a ureteroscope.

FIG. 18 is a hardware and fluid flow block diagram of a ureteroscope.

### DETAILED DESCRIPTION

Implementations of the present disclosure generally relate to biological object removal systems, devices, and related methods. Certain implementations generally relate to devices and systems configured to generate a vacuum, for instance, capable of manipulating, immobilizing, and/or aspirating a solid object and/or debris from an ablated or comminuted solid object in a liquid filled environment. In some cases, the vacuum is generated and/or maintained via a Venturi or Entrainment effect. Configurations described herein may be useful, for example, for capturing, manipulating, immobilizing, and/or removing biological objects from anatomical structures inside the body, such as intact kidney stones, or portions of kidney stones, or debris from ablated kidney stones. For example, some kidney stones may be generally small enough to pass through the ureter of a subject but may be unable to due to, for example, a disease state of the subject. In some cases, the kidney stones are too large or are otherwise to pass through the ureter of the subject. In some cases, the systems and devices described herein include an ablation instrument, such as a laser or ultrasound, configured to ablate one or more solid objects into a plurality of smaller solid objects. Ultrasound can include an ultrasound ablation tool configured to provide ultrasonic lithotripsy. Such systems may be useful, for example, for breaking up kidney stones that are too large to pass through the ureter of a subject. Additionally, or alternatively, the vacuum created by the devices and systems described herein may be useful for aspirating a plurality of smaller solid objects into the device, thus removing it from the liquid filled environment (e.g., the kidney).

Some implementations generally relate to methods of using the systems and devices disclosed herein. For example, the devices and systems described herein may be useful for capturing and/or removing a biological object at a location internal to a subject (such as, from an anatomical structure). Biological objects can include blood clots, tumors, tissue samples, and urinary or fragmented urinary calculi such as bladder stones, ureter stones, and kidney stones.

Some implementations generally relate to methods of using the systems and devices disclosed herein. For example, the devices and systems described herein may be useful for capturing and/or removing a biological object at a location internal to a subject (such as, from an anatomical structure). Biological objects can include blood clots, tumors, tissue samples, and urinary or fragmented urinary calculi such as bladder stones, ureter stones, and kidney stones.

The phrase “location internal to a subject” as used herein generally refers to a cavity, orifice, anatomical structure, or organ within a subject. For example, in some cases, the location internal to the subject is a kidney, a bladder, a heart, a colon, a duodenum, an ileum, a jejunum, a stomach, an esophagus, an intestine, a mouth, a liver, a lung, a pancreas, a spleen, a lymph node, a (blood) vessel, a gland, an ear canal, a urethra, a uterus, a gallbladder, an ovary, or a nasal cavity. In an example set of cases, the location internal to the subject is a kidney or bladder.

The term “subject,” as used herein, refers to an individual organism such as a human or an animal. In some cases, the subject is a mammal (e.g., a human, a non-human primate, or a non-human mammal), a vertebrate, a laboratory animal, a domesticated animal, an agricultural animal, or a companion animal. In some cases, the subject is a human. In some cases, the subject is a rodent, a mouse, a rat, a hamster, a rabbit, a dog, a cat, a cow, a goat, a sheep, or a pig.

The term “effective cross-sectional area” as used herein, refers to a total, cumulative available area. For example, the effective cross-sectional area can be a sum of discrete, or individual areas. The effective cross-sectional area can define the total, cumulative area of a cross-sectional opening. For example, the effective cross-sectional area can be a sum of individual cross-sectional areas of one or more lumens.

In some cases, the articles and systems described herein are administered to a subject. In certain cases, the system may be administered surgically (e.g., inserted), through an incision, typically endoscopically using a catheter or similar, in other cases, the devices may be inserted into the body orally, rectally, vaginally, nasally, or uretherally. In certain cases, the system is administered such that at least a portion of the system accesses a location internal to the subject such as an organ (e.g., the kidney).

In some cases, the system is configured to manipulate a liquid at the location internal to the subject. As used herein, a “liquid” is given its ordinary meaning. A liquid generally cannot maintain a defined shape and will flow during an observable time frame to fill the container in which it is put. Thus, the liquid may have any suitable viscosity that permits flow. If two or more liquids are present, each liquid may be independently selected among essentially any liquids by those of ordinary skill in the art. Typically, the liquid will be sterile water or sterile normal saline or phosphate buffered saline or other osmotically balanced liquid or another IV fluid suitable for use in a human patient.

Implementations of the disclosure are related to devices and systems suitable for use in a surgical procedure at a location internal to a subject. In some cases, the devices are suitable for use at a location internal to a subject at least partially filled with a surrounding liquid (e.g., an organ of the subject).

In some cases, the devices described herein are liquid-jet powered instruments. In some cases, the instruments described herein are Venturi-assisted instruments. In some cases, the instruments described herein are useful for ablation and/or ablation assistance (e.g., of a solid deposit such as a kidney stone). For example, the instruments described herein may be useful for capturing a solid deposit such that it may be ablated (e.g., by an ablation instrument such as a laser, ultrasound probe, or other suitable ablation instruments). As described herein, the liquid-jet powered instruments, Venturi-assisted instruments, and/or other instruments can refer to a catheter, an aspiration catheter, and/or an evacuation catheter.

### Overview

Kidney stones affect approximately 10% of individuals in the United States, with nearly 470,000 surgeries performed annually to relieve symptoms and prevent complications. Current treatment modalities include extracorporeal shock wave lithotripsy (ESWL), ureteroscopy (URS), and percutaneous nephrolithotomy (PCNL). Of these, URS is the most prevalent, accounting for approximately two-thirds of all kidney stone surgeries due to its versatility in treating a broad range of stone sizes using lithotripsy devices to break up stones and baskets to remove fragments and small stones. URS procedures have been steadily increasing by about 15% annually since 2012.

Kidney stone procedures aim to maximize the removal of stones and fragments. Despite advancements in lithotripsy and ureteroscopy technology, stone clearance remains inconsistent, particularly for larger or more complex stones, with stone-free rates (defined as no stones remaining on follow-up CT scan imaging) often declining to 50% in these cases. Residual fragments can lead to symptoms and complications, including acute stone events, regrowth, and infection. Consequently, achieving complete stone clearance is critical to reducing reintervention rates and optimizing long-term outcomes.

There are still necessary improvements in the efficiency and safety of URS, including pressure and temperature management inside the kidney, maintaining a clear visual field, and preventing stone fragments from moving away from operative devices. Enhancing these aspects of URS aims to prevent injury and reduce procedure times.

Recent innovations, such as direct in-scope suction (DISS) and flexible and navigable sheaths (FANS) aim to improve stone-free rates and procedural efficiency, though these technologies have limitations. DISS uses the ureteroscope's small working channel to aspirate dust and small debris, which enhances visibility and helps regulate intrarenal pressure (IRP). However, the narrow diameter of the working channel restricts the removal of larger fragments and causes vacuum pressure loss at the distal end, reducing its ability to effectively clear debris and control retropulsion. This compromised visibility and resulting retropulsion increase the risk of unintended ablation of kidney walls and reduce procedural efficiency. Furthermore, the reduction in suction can lead to an increase in IRP which can have significant consequences (as described below).

Ureteral access sheaths (UAS), including vacuum-assisted FANS models, offer an alternative approach by combining suction with irrigation to maintain low IRP and reduce the “snow globe” effect caused by debris during lithotripsy. FANS provide additional maneuverability, allowing for navigation through renal calyces to suction larger fragments. To use FANS efficiently, high-pressure irrigation is required notably to remove fragments. However, manual control of irrigation and suction often causes kidney distension or collapse during procedures due to difficulties in balancing in and out flow and maintaining IRP. This complexity adds to the surgeon's workload, as they must also manage laser ablation time and wattage to prevent temperature spikes, further diverting their focus and decreasing procedural efficiency, while amplifying the risks associated with managing pressure and temperature Limitations in current laser lithotripsy techniques during URS often lead to prolonged procedures and incomplete stone clearance. Surgeons may attempt mechanical capture and removal of residual fragments with stone retrieval tools, adding time and complexity and requiring coordination with support staff. Alternatively, “dusting” techniques may be employed to break stones into passable particles, though residual dust and small fragments can lead to acute stone events. Limited visibility, often caused by dust clouds and bubbles, hampers precise targeting which leads to residual stone fragments and requires increased irrigation, prolonging the procedure. To prevent thermal injury, surgeons may lower laser wattage or operate it intermittently, which also increases procedure times.

Effective IRP management is essential to prevent complications during ureteroscopy. Although elevated irrigation flow improves visibility during lasing, it can concurrently increase IRP, raising the risk of pyelovenous, pyelolymphatic, and pyelosinus backflow, which can lead to severe outcomes like urosepsis, systemic infection, and kidney damage. These risks not only increase the cost of the procedure due to prolonged hospital stay but also increase the risk of patient death. While UAS and FANS improve fluid outflow to help manage IRP, their use entails risks. Larger-diameter UAS, though effective at reducing IRP, increases the risk of ureteral injury, especially in patients with narrow ureters. One study found that nearly 50% of patients experience ureteral wall damage with UAS over 11 Fr, with the risk rising for even larger sheaths. Although UAS and FANS technologies can control IRP to prevent overpressure and distension, they risk kidney collapse under vacuum suction, leading to bleeding and other complications.

Emerging pressure-sensing ureteroscopes offer real-time IRP monitoring but do not actively control it, leaving pressure regulation in the hands of the surgeon, who must monitor pressure and manually adjust irrigation or use aspiration to lower IRP. This reactive approach relies on the surgeon's ability to interpret and respond promptly to prevent complications.

Temperature management with lasers, particularly Holmium: YAG and Thulium fiber lasers, also presents challenges. Temperatures above 43° C. can cause tissue damage, necessitating techniques like increased or chilled irrigation and reduced laser activation or wattage, which often prolong procedures.

Despite advancements, the current ureteroscopic lithotripsy paradigm challenges surgeons' ability to see, effectively clear stones and fragments, and manage pressure and temperature to safely navigate and treat renal calculi in the upper urinary tract. Existing methods frequently leave fragments behind, resulting in complications for over 40% of patients and requiring re-treatment within a year for up to ⅓ of cases.

The systems and methods described herein aim to address many of these limitations, offering substantial improvements in efficacy, safety, and efficiency. This novel approach has the potential to reduce complications, decrease re-treatment rates, and set a new standard for kidney stone surgery.

### Removal of Biological Objects

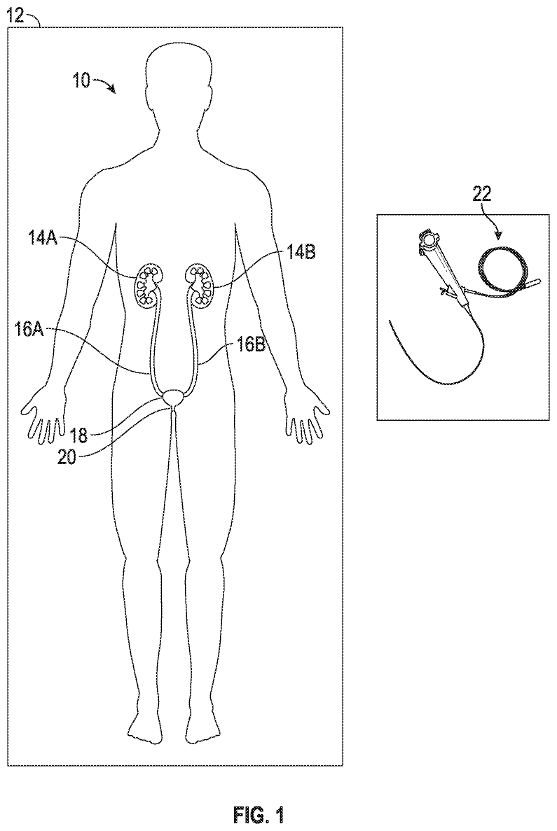

As shown in FIG. 1, a patient 10 can include a first kidney 14A and a second kidney 14B. The patient 10 can further include a first ureter 16A, a second ureter 16B, a bladder 18, and a urethra 20.

The first kidney 14A and the second kidney 14B can be internal organs located within the torso of the patient 10 and positioned below or inferior to the ribcage and laterally on either side of the patient's spine. The first kidney 14A and the second kidney 14B can be configured to process fluids. For example, the first kidney 14A and the second kidney 14B can filter blood, remove waste and extra fluid, balance fluids, and produce hormones and red blood cells. The first kidney 14A and the second kidney 14B can filter fluids resulting in urine. In some cases, the first kidney 14A and/or the second kidney 14B can produce solid deposits or kidney stones. The solid deposits can be formed when the fluids within the first kidney 14A and/or the second kidney 14B include too many crystal-forming substances in relation to the amount of fluid. In some examples, the first kidney 14A and/or the second kidney 14B can include a high concentration of calcium, oxalate, and/or uric acid in relation to the amount of fluid. Accordingly, the fluid is insufficient to dilute the crystal-forming substances and is insufficient to prevent the crystal-forming substances from forming solid deposits.

The first ureter 16A and the second ureter 16B can each be a hollow tube configured to transport fluids. The first ureter 16A and the second ureter 16B can be located in the patient's torso or abdomen.

The bladder 18 can be a hollow, elastic internal organ located in the lower part of a patient's abdomen. The bladder 18 can be configured to collect and store fluids. For example, the bladder 18 can be configured to collect and store urine from the first kidney 14A and/or the second kidney 14B.

The urethra 20 can be a hollow tube configured to transport fluids. The urethra can be in fluid communication with an external environment. Accordingly, the urethra 20 can be configured to expel fluids from the patient's body.

The first kidney 14A and the second kidney 14B can be in fluid communication with the bladder 18 via a corresponding first ureter 16A or second ureter 16B. For example, the first ureter 16A can extend between the first kidney 14A and the bladder 18 and the second ureter 16B can extend between the second kidney 14B and the bladder 18. Accordingly, fluids can be passed from the first kidney 14A and/or the second kidney 14B to the bladder 18 via the corresponding first ureter 16A or second ureter 16B. The bladder can be in fluid communication with an external environment via the urethra 20.

The above identified organs can define the urinary tract or urinary system of a patient. In a healthy patient, fluids such as urine can pass unobstructed through the urinary system. Solid deposits formed within the kidneys can block fluid flow within the urinary system. Blocking the fluid flow can result in a buildup of fluid and pressure causing pain and/or discomfort to the patient. Additionally, in some cases, the solid deposits can have an irregular shape. For example, the solid deposits can have sharp edges. The sharp edges can puncture tissue resulting in additional pain, discomfort, bleeding, and possible infection. To alleviate pain, discomfort, and/or otherwise treat or prevent further risk of injury, methods have been developed to remove solid deposits.

The devices and methods for removing solid deposits from a patient's urinary system can include an operating table 12 and instruments (i.e., devices) including an evacuation tube such as a ureteroscope 22.

The operating table 12 can be a device configured to support and/or restrain a patient during a medical procedure. For example, the operating table 12 may support an unconscious and/or medicated patient 10 for the duration of a medical procedure such as a ureteroscopy.

The ureteroscope 22 can include a thin, tube-shaped shaft. The thin, tube-shaped shaft can have an interior lumen sized to receive at least a portion of a solid deposit. The ureteroscope 22 can be configured to pass through the patient's urinary system to reach the solid deposit (such as, a kidney stone). The ureteroscope 22 can further include a light and a lens to assist a physician in navigating the patient's urinary system and/or identifying solid deposits. The ureteroscope 22 can include a working channel. In some cases, a laser can be placed into the working channel of the ureteroscope 22. The laser can be configured to apply energy to and break up solid deposits. Additionally or alternatively, in some cases, a gripping mechanism can be placed into the working channel of the ureteroscope 22. The gripping mechanism can be configured to secure one or more solid deposits for extraction.

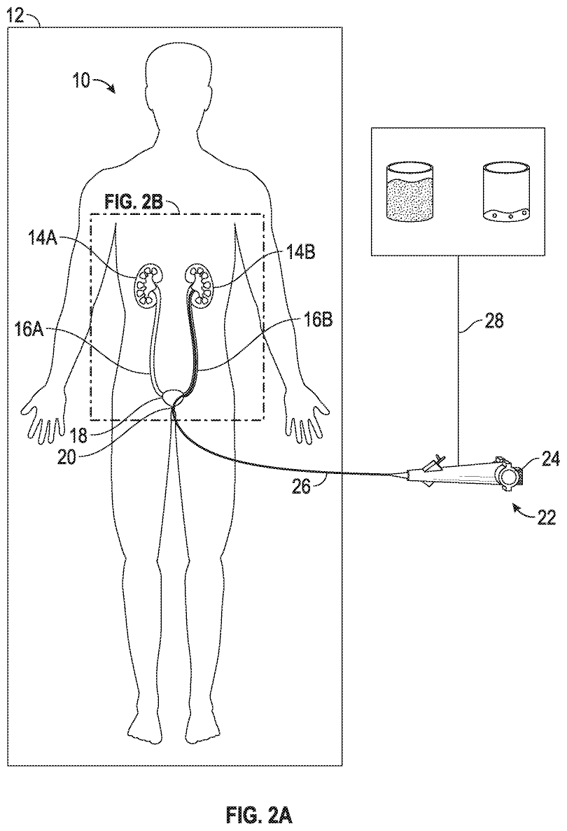

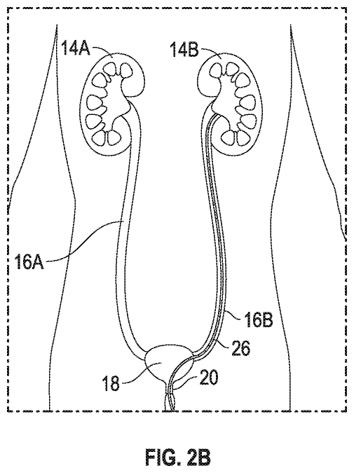

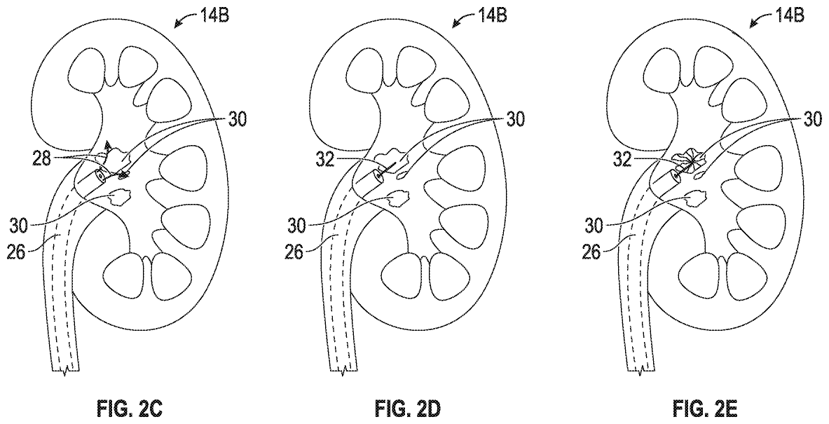

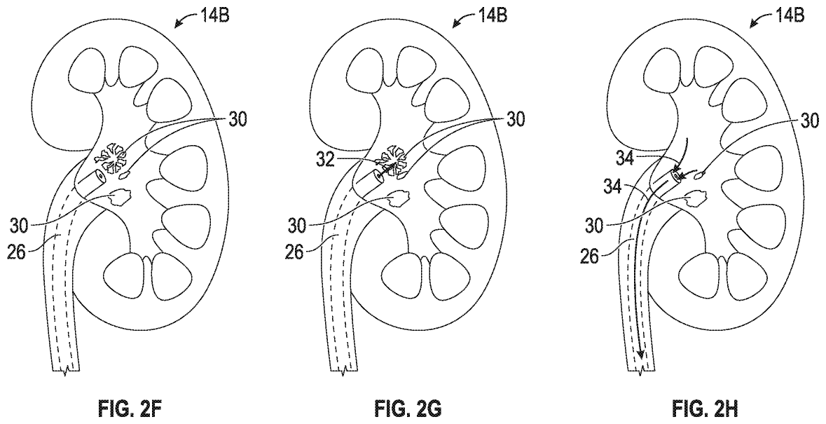

FIGS. 2A-2H illustrate methods and steps of using a ureteroscope 22 to remove solid deposits from a patient's kidney. While FIGS. 2A-2H illustrate the ureteroscope 22 being applied to the second kidney 14B, the approaches described herein can be utilized for removing solid deposits from either kidney and/or anywhere along the urinary system or, more generally, from any location in the body.

As shown in FIG. 2A, a patient 10 can be supported on an operating table 12. In some cases, the patient 10 can be medicated. For example, the patient 10 can be generally and/or locally anesthetized. The ureteroscope 22 can be introduced to the patient's urinary system. For example, the ureteroscope 22 can be introduced via the urethra 20. In some cases, the ureteroscope 22 can include a sheath 26 sized to pass through the patient's urethra 20, bladder 18, ureter 16A/16B and/or kidney 14A/14B. The sheath 26 can be a tube. For example, the sheath 26 can have an outer diameter between about 0.5 and 4 mm, or between about 1.5 and 3.5 mm. In some cases, the sheath 26 can have a length between 40 cm and 100 cm. In some cases, the sheath 26 can have a length between 50 cm and 70 cm. For example, the sheath 26 can be about 60 cm in length. The sheath 26 can include the working channel of the ureteroscope 22. The working channel can extend through the length of the sheath 26. The working channel can have an outer diameter smaller than the outer diameter than the sheath 26. In some cases, the working channel can have an outer diameter between about 1.2 mm and 1.5 mm. The ureteroscope 22 can further include a handle portion 24 and a liquid supply catheter 28 (also referred to as a fluid supply catheter). The handle portion 24 can include an eyepiece or camera for a physician to see for navigation and identification of solid deposits. For example, the handle portion 24 can include a digital complementary metal oxide semiconductor (“CMOS”) camera. The handle portion 24 can be a Y-connector configured to fluidly connect the ureteroscope 22 to a first liquid supply (also referred to as a first liquid source, a first fluid supply, a first fluid source and/or a first fluid reservoir) for irrigation, a second liquid supply (also referred to as a second liquid source, a second fluid supply, a second fluid source and/or a second fluid reservoir) for aspiration, and/or laser or gripping mechanism insertion.

FIG. 2B illustrates a sheath 26 extending through the patient's urinary system and positioned in the second kidney 14B via the second ureter 16B, bladder 18, and urethra 18. The ureteroscope 22 may be generally used to identify and remove solid deposits.

FIG. 2C illustrates a step of inflating the second kidney 14B. As described herein, the ureteroscope 22 can include a sheath 26. The sheath 26 can be in fluid communication with a liquid source. The liquid source can also be referred to as a liquid supply and/or a liquid reservoir. The liquid source can be an input supply of a liquid to be used by a ureteroscope 22 to irrigate and/or flush the patient's urinary system. In some cases, the sheath 26 can introduce a liquid 28 into the second kidney 14B to expand the kidney and enhance visibility. For example, expanding the second kidney 14B can assist a physician in identifying one or more solid deposits 30.

In some cases, the ureteroscope 22 can include an end-effector located at the distal end of the sheath 26. The end-effector can be configured to grasp and secure a solid deposit. After securing the solid deposit, the sheath 26 and/or ureteroscope 22 can be removed from the patient 10. In some cases, the ureteroscope 22 can be in fluid communication with an aspiration source. For example, the ureteroscope 22 can be in fluid communication with a vacuum source. Accordingly, the ureteroscope 22 can be configured to aspirate solid deposits through the sheath 26. In some cases, the solid deposits may be too large to be removed by the ureteroscope 22.

FIG. 2D illustrates a step of applying energy 32 to at least one of the one or more solid deposits 30. In some cases, the energy 32 can be a laser. For example, a laser fiber can extend through the sheath 26 and be configured to emit laser energy toward at least one of the one or more solid deposits 30.

As shown in FIG. 2E-2G, the energy 32 can be sufficient to fracture and/or break up the solid deposits 30 into debris of fragmented solid deposits. The fragmented solid deposits can be smaller than the original solid deposit. For example, the fragmented solid deposits can be reduced to dust particles. Accordingly, the fragments may be sized sufficiently small to be aspirated through the sheath 26. In some cases, the fragments may remain too large to be aspirated through the sheath 26. Accordingly, the energy 32 can be applied to the fragments to further fracture and/or break up the solid deposits 30 as shown in FIG. 2G.

FIG. 2H illustrates a step of aspirating the solid deposits through the sheath 26. The ureteroscope 22 can be withdrawn and removed from the patient 10 after the solid deposits are successfully removed from the patient 10.

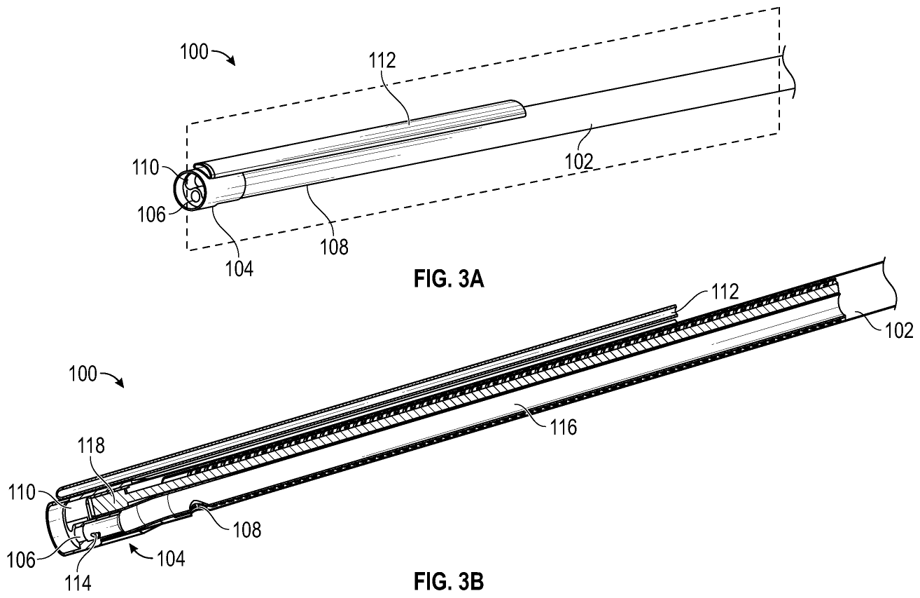

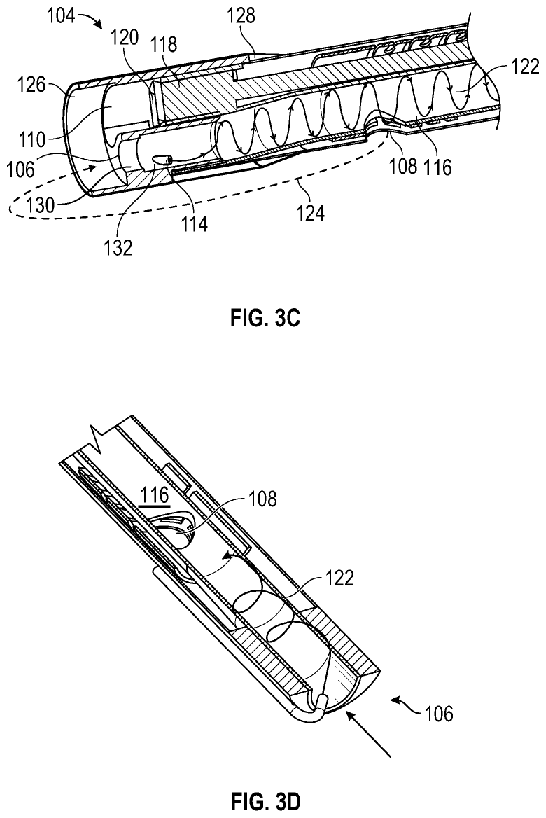

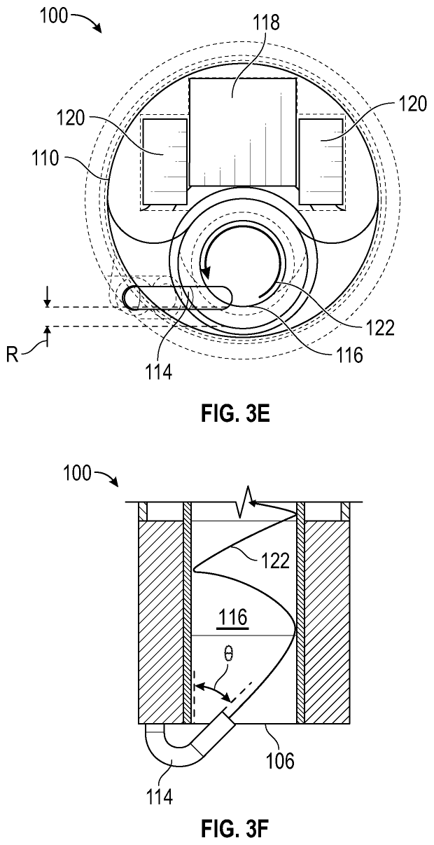

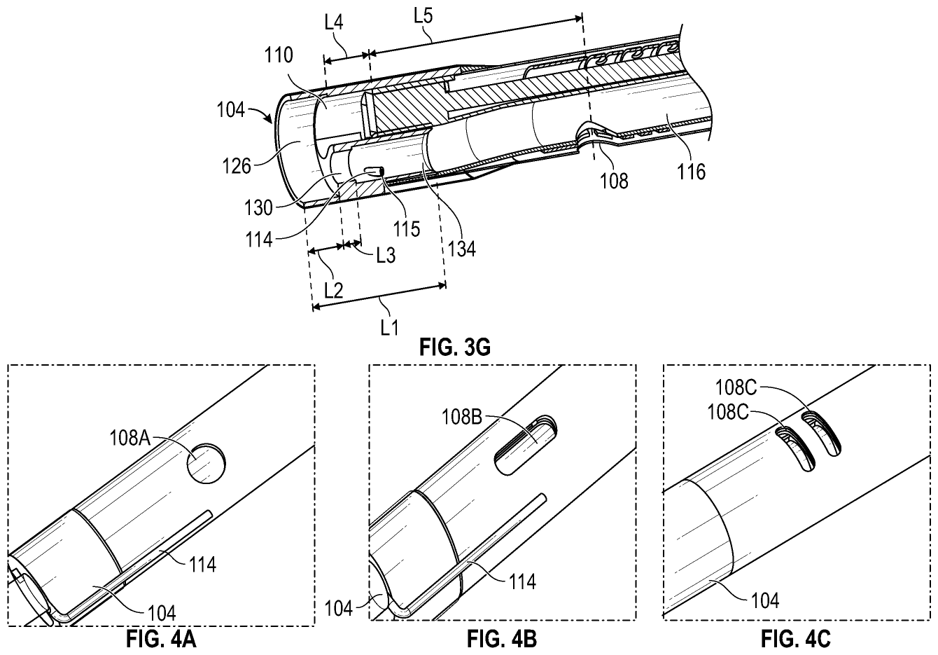

In some implementations, a ureteroscope (sometimes referred to as a device or an endoscopic device) described herein can include a liquid-jet powered aspiration tool or catheter. Such a device will typically include one or more aspiration openings, an evacuation lumen, and one or more vent openings. The one or more aspiration openings are in fluid communication with the evacuation lumen. For example, the one or more aspiration openings can be located along on a sidewall or at a distal end of the evacuation tube. The one or more vent openings are positioned downstream (e.g., relative to the flow of liquid within the evacuation tube, when the device is in operation) of the one or more aspiration openings, as described in more detail herein and shown in FIG. 3A-3F. Those of ordinary skill in the art would understand, based upon the teachings of this specification, that the ureteroscope is generally considered in operation when a liquid is flowing within one or more components of the ureteroscope (e.g., the evacuation lumen, a supply lumen, etc.).

FIGS. 3A-3G illustrate systems and devices for removing a biological object from a location internal to a subject. FIGS. 3A-3B illustrate a ureteroscope 100. FIGS. 3C-3D illustrate cross sectional views of a fluid flow 122 through the ureteroscope 100. FIGS. 3E-3F illustrate a radial distance R and an incidence angle θ between the supply lumen 114 and the evacuation lumen 116, respectively. FIG. 3G illustrates a distal tip 104 of the ureteroscope 100 having various axial lengths.

As shown in FIGS. 3A-3B, a ureteroscope 100 is provided. The ureteroscope 100 can be configured to be inserted within a location internal to a subject and guided to target site located within the anatomy of the subject. The ureteroscope 100 can be configured to remove and/or evacuate the target site of biological objects.

The ureteroscope 100 can include an elongated tubular body 102. The elongated tubular body 102 can include one more lumens extending from a proximal end of the elongated tubular body 102 to a distal end of the elongated tubular body 102. The one or more lumens can be configured to accommodate a fluid flow through the ureteroscope 100 and/or accommodate one or more working devices or tools. In some examples, the one or more lumens can include a supply lumen 114 and an evacuation lumen 116 (also referred to as a working channel).

The supply lumen 114 is a tubular structure extending along the length of the elongated tubular body 102. The supply lumen 114 can provide a liquid jet. In some examples, the supply lumen 114 can be in fluid communication with a fluid source. For example, the supply lumen 114 can be in fluid communication with an irrigation pump configured to supply a fluid flow from the fluid source to the supply lumen 114. The fluid source may be subjected to a pressure for directing the fluid flow distally along the ureteroscope 100 through the supply lumen 114. The supply lumen 114 can redirect the fluid flow from a distal flow to a proximal flow. For example, the supply lumen 114 can include a bend. The supply lumen 114 can be configured to output the fluid into the evacuation lumen 116.

The evacuation lumen 116 is a tubular structure extending along the length of the elongated tubular body 102. The evacuation lumen 116 can aspirate and/or direct fluids and biological objects to an external body for receiving used fluid and evacuated biological objects. The evacuation lumen 116 can have a cross-sectional area extending between a distal end of the elongated tubular body 102 and a proximal end of the elongated tubular body. The cross-sectional area sized and configured to receive the evacuated biological objects.

The ureteroscope 100 can further include a distal tip 104. The distal tip 104 can be provided at the distal end of the elongated tubular body 102. The distal tip 104 can be a working end of the ureteroscope 100 can configured to engage and/or evacuate the target site of biological objects. The distal tip 104 can include an aspiration opening 106. The aspiration opening 106 can be configured to receive the biological objects. The aspiration opening 106 can be a distal most opening of the ureteroscope 100 for introducing the biological object into an interior volume of the ureteroscope 100. In some examples, the distal tip 104 can be formed of an optically clear material. For example, the distal tip 104 can be formed of a polymer (e.g., polycarbonate). In some examples, the distal tip 104 can be formed of a high-performance composite material. In some examples, the distal tip 104 can be formed of a reinforced fluoropolymer (PFA) with ceramic nanoparticles. The PFA with ceramic nanoparticles can provide exceptional laser energy resistance and mechanical durability. In some examples, the distal tip 104 can be formed of jewels. For example, the distal tip 104 can include sapphire, synthetic diamond inserts, fused silica or quartz, spinel, aluminum oxynitride (ALON), fluropolymers (e.g., PFA, FEP, PTFE, ETFE, etc.), borosilicate (e.g., borofloat), chalcognide glasses (e.g., As2S3, AMTIR), fluid glass (e.g., ZBLAN), aluminosilicate glass, cyclic olefin copolymer (COC), etc. The jewels can enhance laser resistance, provide optical clarity, and increase the longevity of the distal tip 104.

The distal tip 104 can include a working opening 110. The working opening 110 can be configured provide a visual field of view. The working opening 110 can allow light to pass through the distal tip 104. Accordingly, light emitted from a light source can illuminate the location internal to the subject. Additionally, a camera can be used to capture images of the location internal to the subject and/or the biological objects positioned within the location internal to the subject.

The elongated tubular body 102 can further include one or more vent openings 108. The one or more vent openings 108 extend through a wall of the ureteroscope 100. As shown in FIG. 3B, the one or more vent openings 108 can extend through a wall of the evacuation lumen 116. The one or more vent openings 108 can be positioned proximally of the distal tip 104. In some examples, the one or more vent openings 108 can be optimized for manufacturing and performance. For example, the one or more vent openings 108 can include a CFD-optimized geometry to maximize fluid entrainment while minimizing flow resistance. In some examples, the one or more vent openings 108 can be laser-cut into the sidewall of the evacuation lumen 116. For example, the one or more vent openings 108 can be laser-cut on an articulation link to maintain robust articulation while maximizing vent size. Additionally and/or alternatively, the one or more vent openings 108 can include a biocompatible hydrophobic coating. For example, the one or more vent openings 108 can include a coating of polydimethylsiloxane (PDMS), polythetrafuoroethylene (PTFE), and Parylene. The coating can be applied around the edges of the one or more vent openings 108 to prevent biological objects from adhering and blocking the evacuation lumen 116.

In some examples, the ureteroscope 100 can further include a secondary lumen 112. The secondary lumen 112 can be an integrated outflow lumen. The secondary lumen 112 can be a dedicated lumen that communicates with the location internal to the subject and can be used to ensure intrarenal pressure (“IRP”) remains in acceptable range and/or for evacuation of fragments of the biological objects. The secondary lumen 112 can be connected to various means to control IRP and flow. In some examples, the secondary lumen 112 can be configured to connect to a peristaltic pump, wall suction, a pressure regulator, a pressure sensor, passive outflow, flow restrictors, and/or constant pressure water column.

Turning to FIG. 3C, the distal tip 104 can further include a cup 126, a choke 130 and a working channel extension 132.

The cup 126 can include an annular wall extending distally away from the distal end of the ureteroscope 100. In some examples, the cup 126 may be a distal extension from the ureteroscope 100. For example, the cup 126 can define an outer wall of the distal tip 104 extending distally from a distal face of the working channel extension 132.

In some examples, the cup 126 can be formed of an optically clear material. For example, the cup 126 can be formed of a resin or polymers such as polycarbonate, polymethylmethacrylate, polyethylene terephthalate, cyclic olefin copolymer (COC), cyclic olefin polymer (COP), fluropolymers, (e.g., perfluoroalkoxy alkane, fluorinated ethylene propylene, polytetrafluoroethylene, ethylene tetrafluroroethylene, silicone (e.g., vinyl methyl silicone, polydimethylsiloxane, etc.), fluorosilicone (FVMQ), thermoplastic polyurethane, etc.

The choke 130 can form one or more channels leading into the evacuation lumen 116. The channel of the choke 130 can constrict a fluid flow between a location internal to a subject and the evacuation lumen 116. For example, the choke 130 can define one or more openings or lumens. Each of the openings or lumens can have a cross-sectional area corresponding to a two-dimensional area available for fluid flow through the choke 130. For example, lumens having a circular cross-section can have an effective cross-sectional area defined by the expression πr², where r is the radius of the lumen, although in various embodiments the lumens may define other cross-sectional shapes aside from circular. Each cross-sectional area of the choke 130 can be smaller than the cross-sectional area(s) of the evacuation lumen 116. Accordingly, the choke 130 can be configured to prevent biological objects sized larger than the one or more openings or lumens of the choke 130 from entering the evacuation lumen 116.

The effective cross-sectional area of the choke 130 can be the same, larger, or smaller than the effective cross-sectional area of the evacuation lumen 116. For example, the sum of the cross-sectional areas of two or more openings or lumens of the choke 130 may be the same, larger, or smaller than the effective cross-sectional area of the evacuation lumen 116. Accordingly, a fluid flow may not be restricted while solid debris that could potentially clog the evacuation lumen 116 are prevented from entering the evacuation lumen 116.

The geometry of the choke 130 can correspond to the geometry of the aspiration opening 106. In some examples, the choke 130 can have the same geometric shape as the aspiration opening. For example, the choke 130 can be a circular shape, a rectangular shape, a triangular shape, or other shape corresponding to the geometry of the aspiration opening 106. The choke 130 may be scaled relative to the aspiration opening 106. For example, the cross-sectional areas of the one or more openings or lumens of the choke 130 may be smaller than the cross-sectional area of the aspiration opening 106. Accordingly, the smallest effective cross-sectional area of the ureteroscope 100 and the distal tip 104 can be defined by the choke 130.

In some examples, the effective cross-sectional area of the choke 130 can be uniform along the length of the choke 130. For example, the choke 130 may have a constant diameter for the entire length of the choke 130 as shown in FIG. 3C. In other examples, effective cross-sectional area of the choke 130 can vary along at least a portion of the length of the choke 130. For example, the choke 130 may taper from a first effective cross-sectional area to a second effective cross-sectional area.

In some examples, the choke 130 can be configured to be thermal resistant and laser resistant (such as, have fluoropolymer thermal and laser resistant properties). For example, the choke 130 can be formed from a jewel (e.g., synthetic sapphire), glass, ceramic, sapphire, syntetic diamond inserts, fused silica or quartz, spinel, aluminum oxynitride (ALON), fluoropolymers (e.g., PFA, FEP, PTFE, ETFE, etc.), borosilicate (e.g., borofloat), chalcogenide glasses (e.g., As2S3, AMTIR), fluid glass (e.g., ZBLAN), aluminosilicate glass, cyclic olefin copolymer (COC), cyclic olefin polymer (COP), etc. Accordingly, aspirated biological objects will be sized to fit through the evacuation lumen 116 without blocking and/or plugging the evacuation lumen 116.

The choke 130 can be positioned along the first axial length L1 of the distal tip 104. In some examples, the choke 130 can be positioned at the aspiration opening 106. For example, a distal face of the one or more lumens of the choke 130 can be the aspiration opening 106. In some examples, the choke 130 may be positioned at a distal end of the working channel extension 132 as shown in FIGS. 3C, 3G, and 5A-8. In some examples, the choke 130 may be positioned be between a distal end and a proximal end of the distal tip 104. For example, the choke 130 may be positioned between the distal end and a proximal end of the working channel extension 132.

The working channel extension 132 can extend distally from the distal end of the evacuation lumen 116 of the ureteroscope 100. The working channel extension 132 can extend the length of the evacuation lumen 116. The working channel extension 132 can include the geometric shape and other properties as the evacuation lumen 116. For example, the working channel extension 132 can include the same cross-sectional area(s) as the evacuation lumen 116. Accordingly, the working channel extension 132 can extend the effective length of the evacuation lumen 116.

As shown in FIGS. 3C-3D, the ureteroscope 100 and the distal tip 104 can be used to remove biological objects from the location internal to the subject. In some examples, the ureteroscope 100 can use one or more vacuum levels to assist with evacuating biological objects from the location internal to the subject. A first vacuum level can be provided at a distal end of the ureteroscope 100 and a second vacuum level can be provided at a proximal end of the ureteroscope 100. The first vacuum level can be used to attract and aspirate fluid from the location internal to the subject, including biological objects, into the distal tip 104 and/or the evacuation lumen 116. The second vacuum level can be used to pull and aspirate fluid from the evacuation lumen 116 into a container.

In an operable state, a proximal end of the supply lumen 114 is in fluid communication with a fluid source via an irrigation pump. A distal end of the supply lumen 114 is in fluid communication with a distal end of the evacuation lumen 116. The distal end of the evacuation lumen 116 is also in fluid communication with the aspiration opening 106. A proximal end of the evacuation lumen 116 is in fluid communication with an aspiration pump.

The irrigation pump can provide a first fluid flow from the fluid source into the supply lumen 114. In some examples, the irrigation pump can provide the first fluid flow into the supply lumen 114 in a first direction. The supply lumen 114 can redirect the first fluid flow from the first direction to a second direction. The first fluid flow can flow from the supply lumen 114 into the evacuation lumen 116 in the second direction. Outputting the first fluid flow from the supply lumen 114 to the evacuation lumen 116 can create the first vacuum level at the distal end of the evacuation lumen 116 using a Venturi effect in which a low pressure region is created to form a vacuum effect. Additionally and/or alternatively, an entrainment effect may be created. The first vacuum level can draw fluid and biological objects from the location internal to the subject into the aspiration opening 106. The fluid flow from the location internal to the subject can be a second fluid flow such as through an entrained fluid flow. Accordingly, introducing the first fluid flow into the evacuation lumen 116 can induce the second fluid flow into the evacuation lumen 116. The first fluid flow and the second fluid flow can flow in the second direction toward the proximal end of the evacuation lumen 116. The aspiration pump can create a second vacuum level at the proximal end of the evacuation lumen 116. For example, the aspiration pump can provide negative pressure at the proximal end of the evacuation lumen 116 to regulate the first fluid flow and the second fluid flow through the evacuation lumen 116. The irrigation pump and the aspiration pump can be used to regulate fluid flows, pressures, and temperatures within the location internal to the subject and the ureteroscope 100. For example, the irrigation pump and the aspiration pump can be controlled to change their operating states to change relative flow rates.

As further shown in FIGS. 3C-3D, the fluid flow 122 can flow at least partially in the second direction from the distal end of the evacuation lumen 116 to the proximal end of the evacuation lumen 116. The fluid flow 122 can be a combination of the first fluid flow and the second fluid flow. For example, the fluid flow 122 can include a medical fluid from the first fluid flow and biological fluids from the location internal to the subject. In some examples, the fluid flow 122 can form a helical flow path or it can be a vortex around the inside of the evacuation lumen 116. A vortex can maximize mixing the first fluid flow and the second fluid flow. Mixing the first fluid flow and the second fluid flow can maximize inflow through the distal tip 104.

The one or more vent openings 108 can be configured to recirculate at least a portion of the second fluid flow from the evacuation lumen 116 into the location internal to the subject. Recirculating the fluid can regulate pressures and temperatures within the location internal to the subject. Accordingly, the ureteroscope 100 may be continuously used for the duration of the procedure without intermittently waiting for pressure to build up in the location internal to the subject and/or for temperatures to cool. In some examples, the one or more vent openings 108 can prevent aspirated biological objects from recirculating into the location internal to the subject. For example, the one or more vent openings 108 can be sized to allow fluid to flow through the one or more vent openings 108 but prevent solid debris from flowing through the one or more vent openings 108.

As shown in FIG. 3E, the distal tip 104 can include an optical sensor 118 and one or more light sources 120. The optical sensor 118 can be an imaging device. For example, the optical sensor 118 can be configured to generate an image. In some examples, the optical sensor 118 can be a visual light imaging device (e.g., a camera, CCD imager, CMOS imager, optical fiber, etc.), an x-ray imaging device, and/or an ultrasonic imaging device. For example, the optical sensor 118 can be a CMOS camera. The one or more light sources 120 can be an LED light source.

The distal tip 104 can be integrated and/or an add-on device. For example, the distal tip 104 can be built into the ureteroscope 100. Alternatively, the distal tip 104 can be added as an after-market modification to an existing ureteroscope. In such examples, the distal tip 104 can be a two-piece variation that can be secured to the distal end of the ureteroscope 100 via a securing device 128. In some examples, the securing device 128 can be a heat-shrink tubing.

FIGS. 3C-3G illustrate the distal tip 104 and relative geometries, spacing, and orientations of various components of the ureteroscope 100 and the distal tip 104. The various dimensions and lengths within the distal tip 104 can be optimized to maximize device performance. For example, the various dimensions and lengths can maximize inflow through the aspiration opening 106.

As shown in FIGS. 3C and 3E, the supply lumen 114 can be spacially oriented about a radial distance R and an incidence angle θ relative to the evacuation lumen 116.

The supply lumen 114 can be positioned relative to the evacuation lumen 116. Contributing geometries can include the radial distance R from the outlet 115 of the supply lumen to the inner surface of the evacuation lumen 116. The radial distance R can impact the linear flow path of the first fluid flow from the outlet 115 to the inner surface of the evacuation lumen 116. In some examples, the radial distance R from the outlet 115 of the supply lumen 114 to the inner surface of the evacuation lumen 116 can be a value between 0.0 or about 0.0 mm and 0.5 or about 0.5 mm. In some examples, the radial distance R from the outlet 115 of the supply lumen 114 to the inner surface of the evacuation lumen 116 can be a value between 0.1 or about 0.1 mm and 0.4 or about 0.4 mm. As shown in FIGS. 3D and 3F, the supply lumen 114 can have a bend, also referred to as a J-bend. The bend can be near the wall of the evacuation lumen 116. If the bend is too close, entrainment can be reduced due to the proximity of the bend with the wall. By comparison, if the bend is too far, entrainment is also reduced due to reduced vortex action.

The incidence angle θ can affect the volume of fluid aspirated through the distal tip 104. In some examples, the incidence angle θ can be at an angle configured to create the vortex around the inside of the evacuation lumen 116. As described herein with reference to FIGS. 3C-3D, creating a vortex around the inside of the evacuation lumen 116 can maximize mixing the tip inflow fluid (second fluid flow) and the fluid flow from the supply lumen 114 (first fluid flow). Mixing the fluids can maximize inflow through the distal tip 104. In some examples, the incidence angle θ can be a value between 10 or about 10 degrees and 60 or about 60 degrees. In some examples, the incidence angle θ can be a value between 35 or about 35 degrees and 55 or about 55 degrees. For example, the incidence angle θ can be 45 or about 45 degrees. As shown in the top view of the distal end of the ureteroscope 100 (distal tip 104 is not shown) in FIGS. 3D and 3F, the bend of the supply lumen 114 can direct a liquid jet into the inner surface of the evacuation lumen 116.

Accordingly, as shown in FIGS. 3C-3F, a fluid flow 122 can flow from the supply lumen 114 through the evacuation lumen 116. The fluid flow 122 can be a helical flow extending around the inner surface of the evacuation lumen 116. For example, the fluid flow 122 can be outputted as a liquid jet from an outlet 115 of the supply lumen until the fluid flow 122 contacts the inner surface of the evacuation lumen 116. The fluid flow 122 can then follow the contour of the inner surface of the evacuation lumen 116 to the proximal end. The fluid flow 122 can create a Venturi and/or Entrainment effect at the aspiration opening 106. A recirculation flow 124 can be configured to pass from the fluid flow 122 through the one or more vent openings 108 toward the aspiration opening 106.

As shown in FIG. 3G, the geometry of the components of the distal tip 104 can affect the fluid flow 122. As described above, contributing geometries can include the radial distance R from the outlet 115 of the supply lumen to the inner surface of the evacuation lumen 116 and the incidence angle θ of the supply lumen 114. Other contributing geometries can include a first axial length L1 of the distal tip, a second axial length L2 of the cup, a third axial length L3 of the choke, a fourth axial length L4 of the inner tube length, a the fifth axial length L5 of a proximal offset of the one or more vent openings 108 from the distal end of the ureteroscope 100. The various dimensions and lengths within the distal tip 104 can be optimized to maximize device performance. For example, the various dimensions and lengths can maximize inflow through the aspiration opening 106.

The first axial length L1 can extend from a distal end of the distal tip 104 to a proximal end of the distal tip 104. Accordingly, the distal tip 104 can extend the effective length of the ureteroscope 100 by the first axial length L1.

The second axial length L2 of the cup 126 can be optimized to grasp biological objects while not obstructing the field of view of the optical sensor 118. Grasping biological objects can be improved by increasing the second axial length L2 of the cup 126. However, increasing the second axial length L2 of the cup 126 can impede or obstruct the field of view of the optical sensor 118. In some examples, the second axial length L2 of the cup can be a value between 0.1 or about 0.1 mm and 2.0 or about 2.0 mm. For example, the second axial length L2 of the cup can be a value between 0.5 or about 0.5 mm and 1.5 or about 1.5 mm.

The third axial length L3 of the choke 130 can be minimized to reduce clogging while remaining long enough to be robust. A reduced diameter of the choke 130 can ensure that any stone fragment that passes through are small enough to continue up the working channel and out of the device. In some examples, the third axial length L3 of the choke can be a value between 0.1 or about 0.1 mm and 1.0 or about 1.0 mm. For example, the third axial length L3 of the choke can be a value between 0.2 or about 0.2 mm and 0.5 or about 0.5 mm.

The fourth axial length LA of the working opening 110 can be selected to optimize visualization of an ablation device and biological objects. The length LA can affect the ability for the camera to visualize an ablation device and potential clogs. In some examples, the fourth axial length LA of the working opening 110 can be a value between 0.5 or about 0.5 mm and 2.0 or about 2.0 mm.

The fifth axial length L5 of the proximal offset of the one or more vent openings 108 from the distal end of the ureteroscope 100 can be selected to balance back pressure and vacuum. The fifth axial length L5 of the proximal offset of the one or more vent openings 108 from the distal end of the elongated tubular body 102 can affect the effective length of the Venturi and/or Entrainment effect. A shorter fifth axial length L5 can reduce the distance over which Venturi and/or Entrainment occurs. However, extending the fifth axial length L5 can contribute to back pressure. Both of which can reduce the fluid inflow through the aspiration opening 106. In some examples, the fifth axial length L5 of the proximal offset of the one or more vent openings 108 from the distal end of the ureteroscope 100 can be a value between 2.0 or about 2.0 mm and 10.0 or about 10.0 mm.

The axial length of the working channel extension 132 can be the length of the distal end without the cup 126. Accordingly, the axial length of the working channel extension 132 can be defined as the first axial length L1 minus without the second axial length L2.

In addition to axial lengths described above, various elements of the ureteroscope 100 can include transverse lengths. In some examples, each of the flow paths (e.g., lumens, channels, passageways and openings) can include a transverse length defining at least a portion of an effective cross-sectional area. The transverse lengths can correspond to a length extending orthogonally to a longitudinal axis of the ureteroscope 100. As shown in FIGS. 3A-3G, one or more of the aspiration opening 106, the working opening 110, the supply lumen 114, the evacuation lumen 116, the cup 126, the choke 130, and the working channel extension 132 can include a transverse length.

The transverse lengths can correspond to a dimension of a cross-sectional area of the corresponding flow paths. For example, for circular flow paths, the transverse length can be a radial length extending radially outward from the longitudinal axis of the corresponding flow path. For noncircular flow paths such as a rectangular cross-section, the transverse length can be a horizontal/base length or a vertical/height length. The above description relates to two non-limiting examples of transverse lengths. Other cross-sectional shapes are possible.

The transverse lengths of the ureteroscope 100 can limit a maximum particle size that can be removed by the ureteroscope 100. For example, the maximum particle size that can traverse the evacuation lumen 116 can be determined by a minimum transverse length. Other factors can further limit the maximum particle size that can be removed by the ureteroscope 100. For example, an ablation device may be inserted through the evacuation lumen 116. In such cases, the maximum particle size that can traverse the evacuation lumen 116 can be determined by a transverse gap between the ablation device and an inner surface of the ureteroscope 100. The inner surface of the ureteroscope can be an inner surface of the aspiration opening 106, of a restriction/constriction along the working channel extension 132, of the choke 130, and/or of the evacuation lumen 116. For example, as shown in FIG. 3C, the maximum particle size can be limited by a radial gap between an ablation device (not shown) and the aspiration opening 106/choke 130.

For flow areas having a circular cross-sectional area where the ureteroscope 100 does not have a centering structure, an ablation device may be disposed eccentrically, up to a position along a wall of the flow area. For example, the ablation device may be positioned along a side wall of the flow area. In such cases, the maximum particle size may be limited by an area defined by the diameter of the flow area less the outer diameter of the ablation device. Accordingly, the maximum particle size that can traverse the ureteroscope 100 with an eccentrically disposed ablation device can be twice the size of the maximum particle size that can traverse the ureteroscope 100 with a centrally disposed ablation device.

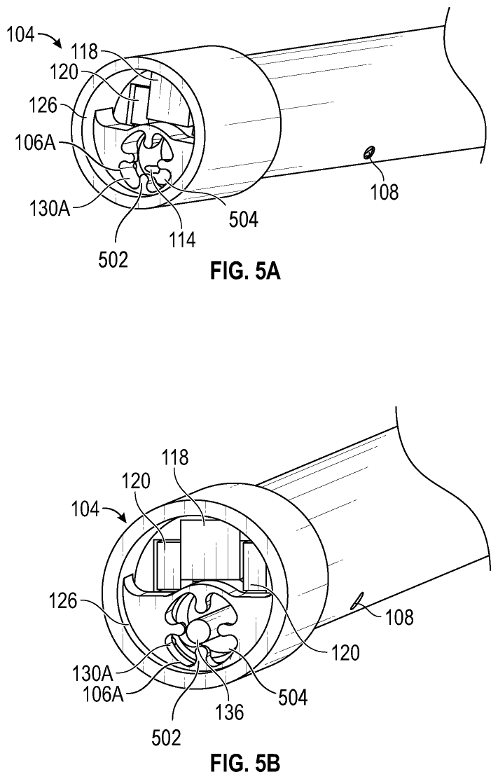

For flow areas having a noncircular cross-sectional area, the maximum particle size that can traverse the ureteroscope 100 can be defined by a maximum transverse gap. For example, as shown in FIG. 5B, a maximum particle size can be defined by the maximum transverse gap distance from the ablation device 136 to the clover shaped aspiration opening 106A.

The evacuation lumen 116 can be sized and configured to receive an ablation device and to provide a sufficient cross-sectional area for removing debris and fluid. In some examples, the ablation device introduced through the evacuation lumen 116 can have an outer diameter of 250 micron or more. For example, the evacuation lumen 116 may accommodate ablation devices having an outer diameter (including a core, cladding, and jacketing material) of 270 or about 270 micron (0.27 or about 0.27 mm) to 500 or about 500 micron (0.5 or about 0.5 mm). In some examples, the evacuation lumen 116 can have a first radial length of 0.5 mm or more. For example, the first radial length can be between 0.5 or about 0.5 mm and 1.0 or about 1.0 mm. In some examples, the first radial length can be between 0.5 or about 0.5 mm and 0.75 or about 0.75 mm. For example, the evacuation lumen 116 can have a first radial length of 0.6 or about 0.6 mm. The evacuation lumen 116 can have a diameter twice the length of the first radial length.

The choke 130 can have a second radial length. The second radial length of the choke 130 can be sized relative to the first radial length of the evacuation lumen 116. In some examples (e.g., when the choke 130 has a circular flow path), the second radial length of the choke 130 can be sized to be 65%-80% of the first radial length of the evacuation lumen 116. For example, the second radial length of the choke 130 can be 75% or about 75% of the first radial length of the evacuation lumen 116. Accordingly, the second radial length can be between 0.4 or about 0.4 mm and 0.5 or about 0.5 mm. For example, the choke 130 can have a second radial length of 0.45 or about 0.45 mm. The choke 130 can have a diameter twice the length of the second radial length. In some examples (e.g., when the choke 130 has a noncircular flow path), the second radial length of the choke 130 can be sized so that the cross-sectional flow area is 45%-60% of the cross-sectional flow area resulting from the first radial length of the evacuation lumen 116. For example, the second radial length of the choke 130 can be sized so that the cross-sectional flow area is 55% or about 55% of the cross-sectional flow area resulting from the first radial length of the evacuation lumen 116.

In some examples, the ureteroscope 100 can include the evacuation lumen 116 having a circular cross-sectional area with first radial length of 0.6 or about 0.6 mm, and diameter of 1.2 or about 1.2 mm, and a choke 130 having a circular cross-sectional area with a second radial length of 0.45 or about 0.45 mm, and diameter of 0.9 or about 0.9 mm. In such examples, the ureteroscope 100 may accommodate a maximum particle size of 440 mirons as defined by the choke diameter (e.g., 0.9 or about 0.9 mm) less the outer diameter of an ablation device (e.g., 0.46 or about 0.46 mm). Accordingly, the maximum particle size of a biological object that can pass through the choke 130 is smaller than the evacuation lumen 116.

Thus, the potential for clogging can be minimized by limiting the maximum particle size that can pass through the evacuation lumen 116. However, the volume of fluid drawn into the aspiration opening 106 and corresponding suction at the distal tip 104 can also be limited.

Sizing the choke 130 relative to the evacuation lumen 116 can balance the effects of mitigating clogging within the evacuation lumen 116 and limiting the second fluid flow. For example, in examples having a circular flow path, the choke 130 may be sized to have a diameter between 65% and 80% of the diameter of the evacuation lumen 116. For noncircular designs, the choke 130 may be sized to have a flow area between 45% and 60% of the flow area of the evacuation lumen 116.

The working opening 110 can be sized and configured to receive the optical sensor 118 and to provide a sufficient cross-sectional area for a field of view for the optical sensor 118. In some examples, the working opening 110 can have a third transverse length of 0.33 mm or more. For example, the third radial length can be between 0.33 or about 0.33 mm and 1.0 or about 1.0 mm. In some examples, the third transverse length can be between 0.65 or about 0.65 mm and 1.0 or about 1.0 mm. In some examples, the working opening 110 can be circular. In such examples, the third transverse length can be a radius between 0.33 or about 0.33 mm and 0.5 or about 0.5 mm, or a diameter between 0.65 or about 0.65 mm and 1.0 or about 1.0 mm. In some examples, the woring opening 110 can be noncircular. In such examples, the third transverse length can be a rectangle having a height and a length each sized between 0.65 or about 0.65 mm and 1.0 or about 1.0 mm.

The cup 126 can be sized relative to one or more components of the ureteroscope 100. For example, the cup 126 can be sized relative to the evacuation lumen 116. Accordingly, the cup 126 can have a fourth transverse length sized between 0.5 or about 0.5 mm and 1.0 or about 1.0 mm. In other examples, the cup 126 can be sized relative to the ureteroscope 100. Accordingly, the cup 126 can be sized to have a diameter between 1.2 or about 1.2 and 2.0 or about 2.5 mm. For example, the cup 126 can be sized to have a diameter of 1.25 or about 1.25 mm FIGS. 4A-4C illustrate various examples of the one or more vent openings 108 described herein with reference to FIGS. 3A-3F. The variety of geometries of the one or more vent openings 108 may be used for the recirculation flow 124. In some examples, the cross sections can be at least as large as the cross section of the working channel 134 to maximize flow through the evacuation lumen 116 and minimize the risk of clogging.

FIG. 4A illustrates an example of a vent opening 108A. The vent opening 108A can be a single circular or round vent opening. In some examples, the vent opening 108A can have a diameter between 1.0 or about 1.0 and 1.5 or about 1.5 mm.

FIG. 4B illustrates an example of a vent opening 108B. The vent opening 108B can be a single longitudinal slot. For example, the vent opening 108B can extend through the sidewall of the evacuation lumen in the axial direction of the ureteroscope 100. The vent opening 108B can have a first dimension and a second dimension. The first dimension can be orthogonal to the longitudinal axis of the ureteroscope. The second dimension can be parallel to the longitudinal axis of the ureteroscope. The second dimension can be greater than the first dimension. In some examples, the first dimension can be a value between 0.4 or about 0.4 and 0.8 or about 0.8 mm. In some examples, the second dimension can have a value between 1.0 or about 1.0 and 2.0 or about 2.0 mm.

FIG. 4C illustrates an example of a plurality of vent port openings 108C. The plurality of vent port openings 108C can each be a slot arranged orthogonally to the longitudinal axis of the ureteroscope 100. Each of the plurality of vent port openings 108C can have a first dimension and a second dimension. The first dimension can be orthogonal to the longitudinal axis of the ureteroscope 100. The second dimension can be parallel to the longitudinal axis of the ureteroscope 100. The first dimension can be greater than the second dimension. In some examples, the first dimension can be a value between 0.4 or about 0.4 and 0.8 or about 0.8 mm. In some examples, the second dimension can have a value between 1.0 or about 1.0 and 2.0 or about 2.0 mm. The ureteroscope 100 can have any quantity of the plurality of vent port openings 108C. For example, the ureteroscope can have 1, 2, 3, 4, or more vent port openings 108C.

FIGS. 5A to 9 illustrate various examples of the choke 130. Common features of all chokes described herein can include a constriction to prevent solid objects from passing the choke 130 that could get stuck and occlude the evacuation lumen 116.

FIGS. 5A-5B illustrate an example of a distal tip 104 having a choke 130A with an aspiration opening 106A. The choke 130A can be a variation of the choke 130 described herein with reference to FIGS. 3A-3G. As shown in FIGS. 5A-5B, the choke 130A can be clover shaped. For example, the clover shape can be a four clover-shaped opening. Accordingly, the choke 130A can include a plurality of fingers 502 extending radially inward from the radially outer circumference of the aspiration opening 106A. The plurality of fingers 502 can define and separate individual leaf openings 504. Each leaf opening 504 can include a cross-sectional area. The effective cross-sectional area of the choke 130A can be a sum of the cross-sectional areas of all of the leaf openings 504. In some examples, plurality of fingers 502 may not connect. Accordingly, as shown in FIGS. 5A-5B, the leaf openings 504 may not be fully separated. Instead, a passage may connect one or more of the leaf openings 504. In such examples, the effective cross-sectional area of the choke 130A may also include the cross-sectional area of the passage. The plurality of fingers 502 can keep an ablation device 136 centered in the evacuation lumen 116 as shown in FIG. 5B. Accordingly, the effective cross-sectional area of the aspiration opening 106A can be maximized while preventing large particles from passing through the choke 130A such that the choke 130A can provide for a maximum inflow while preventing clogging downstream.

The ablation device 136 can be any device configured to break up biological objects. In some examples, the ablation device 136 can provide energy such as laser energy, ultrasound energy, or mechanical energy. In some examples, the ablation device 136 can be coupled to an automated reciprocation mechanism configured to move the ablation device 136 relative to the choke 130. For example, the automated reciprocation mechanism can move the ablation device 136 longitudinally 0.1 or about 0.1 mm to 2.0 or about 2.0 mm. In some examples, the automated reciprocation mechanism can move the ablation device 136 longitudinally 0.5 or about 0.5 mm to 2.0 or about 2.0 mm. The automated reciprocation mechanism can dislodge fragments, prevent fragment accumulation and ensure continuous patency of the outflow path. The automated reciprocation mechanism can include a reciprocating laser bridge assembly. The reciprocating laser bridge assembly can utilize precision-driven piezoelectric actuators or brushless DC motors for silent, smooth, and repeatable reciprocation with minimal vibration. The reciprocation mechanism could also be performed manually by a user via a user interface (e.g., button press or lever movement). Additionally and/or alternatively, the automated reciprocating mechanism can include an automated control. The automated control can be a software that proactively manages reciprocation based on real-time fragment accumulation. In some examples, the automated reciprocation mechanism can be operated according to a wave pattern. The wave pattern can be a pattern of longitudinally moving the ablation device 136. For example, the wave pattern can be a continuous motion of moving the ablation device 136 between a proximal position and a distal position. In some examples, the wave pattern can correspond to the energy output of the ablation device 136.

The ablation device 136 can include certain safety and efficiency features. In some examples, the ablation device 136 can incorporate intelligent safety and optimization controls. For example, the ablation device 136 can inhibit automated activation to prevent firing the ablation device when incorrectly positioned or aimed at non-target structures. In some examples, the ablation device 136 can include a dynamic output optimization which may use an artificial intelligence (AI) to adjust settings based on fragment composition and position to maximize fragmentation efficiency as described in greater detail herein with reference to FIG. 18. In some examples, the ablation device 136 can modulate an adaptive pulse to reduce collateral tissue damage by tailoring pulses to fragment properties. In some examples, the ablation device 136 can optimize energy power to minimize the risk of ureteral injury.

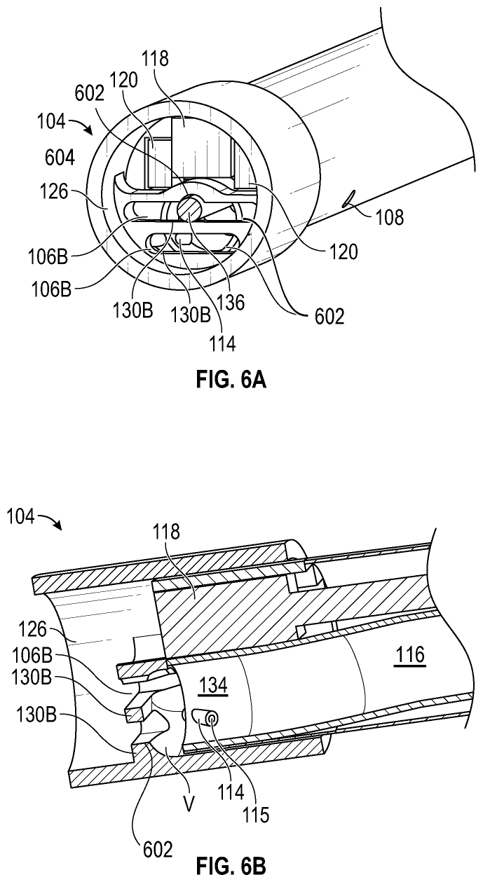

FIGS. 6A-6B illustrate an example of a distal tip 104 having a choke 130B with an aspiration opening 106B. The choke 130B can be a variation of the choke 130 described herein with reference to FIGS. 3A-3F. As shown in FIGS. 6A-6B, the choke 130B can include a plurality of slots 602. The plurality of slots 602 can define and separate individual transverse openings. Each slot 602 can include a cross-sectional area. The effective cross-sectional area of the choke 130B can be a sum of the cross-sectional areas of all of the slots 602. The choke 130B can provide a recess 604 to accommodate an ablation device 136. The recess 604 can keep the ablation device 136 centered in the working channel 134. The cross sectional area(s) of the choke 130B can be maximized while preventing large particles from passing through the choke 130B. Additionally, a volume V can be present proximal of the choke 130B and distal of the outlet 115 of the supply lumen 114. The volume V can maximize the inflow while restricting particle size.

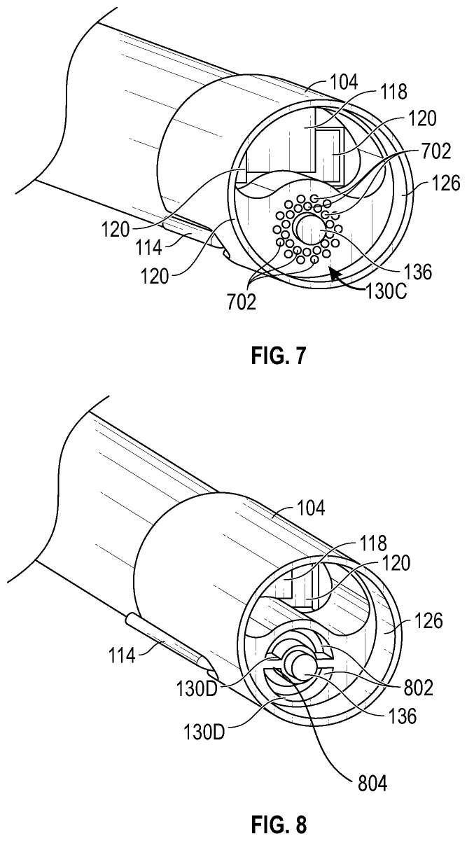

FIG. 7 illustrates an example of a distal tip 104 having a choke 130C with a plurality of aspiration openings 702. The choke 130C can be a variation of the choke 130 described herein with reference to FIGS. 3A-3F. As shown in FIG. 7, the choke 130C can include a plurality of aspiration openings 702 for receiving biological objects. The plurality of aspiration openings 702 can be arranged annularly and radially. The plurality of aspiration openings 702 can define and separate individual aspiration openings. Each aspiration opening 702 can include a cross-sectional area. The effective cross-sectional area of the choke 130C can be a sum of the cross-sectional areas of all of the aspiration openings 702. The choke 130C can include another opening to accommodate an ablation device 136. The other opening can keep the ablation device 136 centered in the working channel 134. The summation of the cross-sectional area(s) of the plurality of aspiration openings 106C can be maximized while the choke 130C prevents large particles from passing through the choke 130C.

FIG. 8 illustrates an example of a distal tip 104 having a choke 130D with two hemispheric aspiration openings 802. The choke 130D can be a variation of the choke 130 described herein with reference to FIGS. 3A-3F. As shown in FIG. 8, the two hemispheric aspiration openings 802 can receive biological objects. The choke 130C can separate the two hemispheric aspiration openings 802 with a divider 804. In some examples, the divider 804 can accommodate an ablation device 136. The divider 804 can keep the ablation device 136 centered in the working channel 134. The summation of the cross-sectional area(s) of the two hemispheric aspiration openings 106D can be maximized while the divider prevents large particles from passing through the choke 130D.

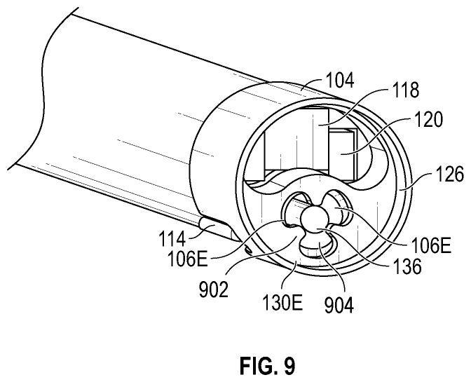

FIG. 9 illustrates an example of a distal tip 104 having a choke 130E with an aspiration opening 106E. The choke 130E can be a variation of the choke 130 described herein with reference to FIGS. 3A-3F. As shown in FIG. 9, the choke 130E can be clover shaped. For example, the clover shape can be a three clover-shaped opening. Accordingly, the choke 130E can include a plurality of fingers 902 extending radially inward from the radially outer circumference of the aspiration opening 106E. The plurality of fingers 902 can define and separate individual leaf openings 904. Each leaf opening 904 can include a cross-sectional area. The effective cross-sectional area of the choke 130E can be a sum of the cross-sectional areas of all of the leaf openings 904. In some examples, plurality of fingers 902 may not connect. Accordingly, as shown in FIG. 9, the leaf openings 904 may not be fully separated. Instead, a passage may connect one or more of the leaf openings 904. In such examples, the effective cross-sectional area of the choke 130E may also include the cross-sectional area of the passage. The plurality of fingers can keep an ablation device 136 centered in the working channel 134. Additionally, the cross-sectional area of the aspiration opening 106E can be maximized while preventing large particles from passing through the choke 130E. Accordingly, the choke 130E can provide for a maximum inflow while preventing clogging downstream.

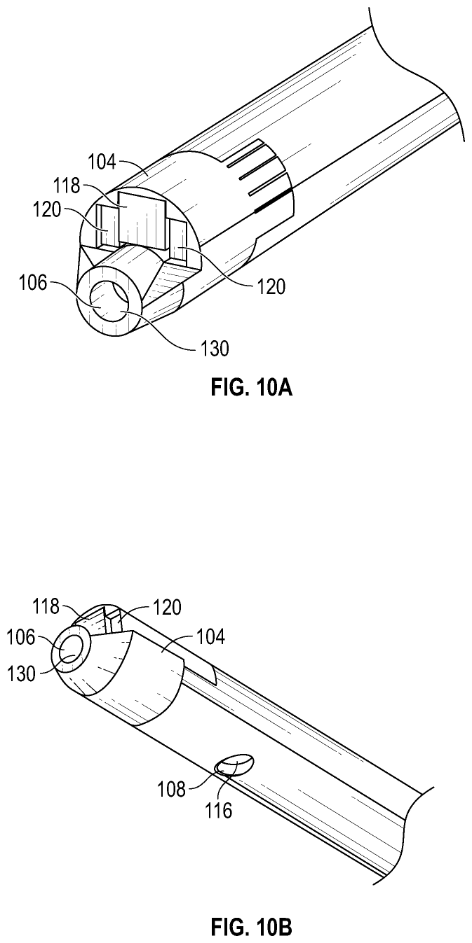

FIGS. 10A-10B illustrate an example of a distal tip 104 without a cup. As shown in FIGS. 10A-10B, the distal tip 104 may not provide any structure distal of the optical sensor 118 or the light sources 120. As shown in FIGS. 10A-10B, the aspiration opening 106 can be a distal most opening and the choke 130 can define a constricted lumen relative to the evacuation lumen 116.

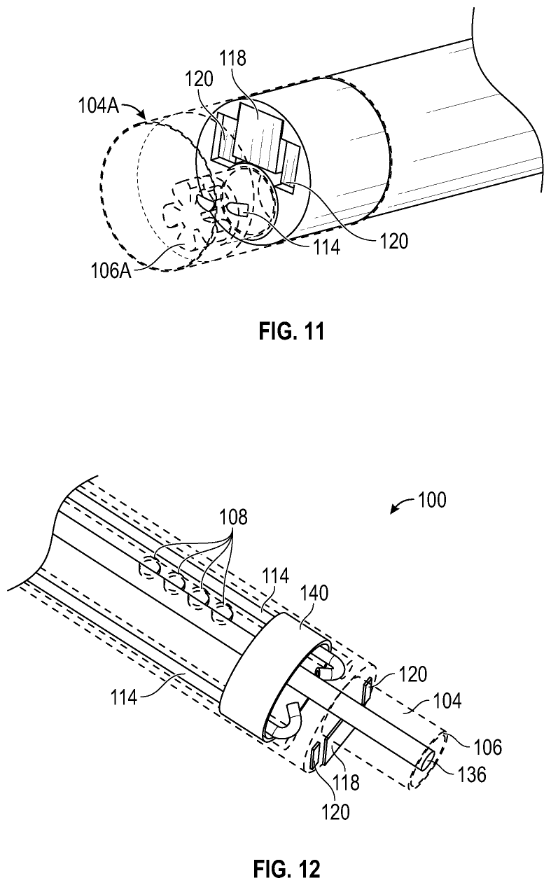

FIG. 11 illustrates an example distal tip 104A. The distal tip 104A can be a variation of the distal tip 104 described herein with reference to FIGS. 3A-3F. The distal tip 104A may not include the cup 126 described above. Instead, the distal tip 104 can be a solid optically transparent device extending distally from a distal end of the ureteroscope 100. The solid optically transparent distal tip can function as a “windshield.” In some examples, the distal tip 104A can be coupled to the ureteroscope 100 by a securing device such as (PET heat shrink). The distal tip 104A can have one of a variety of different choke designs. For example, the distal tip 104A can include an aspiration opening 106A that is clover shaped. As shown in FIG. 11, the clover shape can be a five clover shape having five fingers separating leave openings.

FIG. 12 illustrates a bottom view of a ureteroscope 100. As shown in FIG. 12, the ureteroscope 100 can include multiple supply lumens 114. In some examples, the ureteroscope 100 can include 1, 2, 3, 4, or more supply lumens 114. The supply lumens 114 can be secured in place via a ring 140. The ring 140 can be welded to the supply lumens 114. In some examples, the supply lumens 114 can extend in lumens and terminate to a deflection mechanism located in a handle of the ureteroscope 100. The deflection mechanism can include pull wires for deflecting the distal end of the ureteroscope 100. Combining the liquid jet (driving fluid from the supply lumen 114) with pull wires can allow for more efficient space utilization in the shaft of the ureteroscope 100. As further illustrated, the ureteroscope 100 can include a plurality of vent openings 108. The plurality of vent openings 108 can be any of the variations described herein.

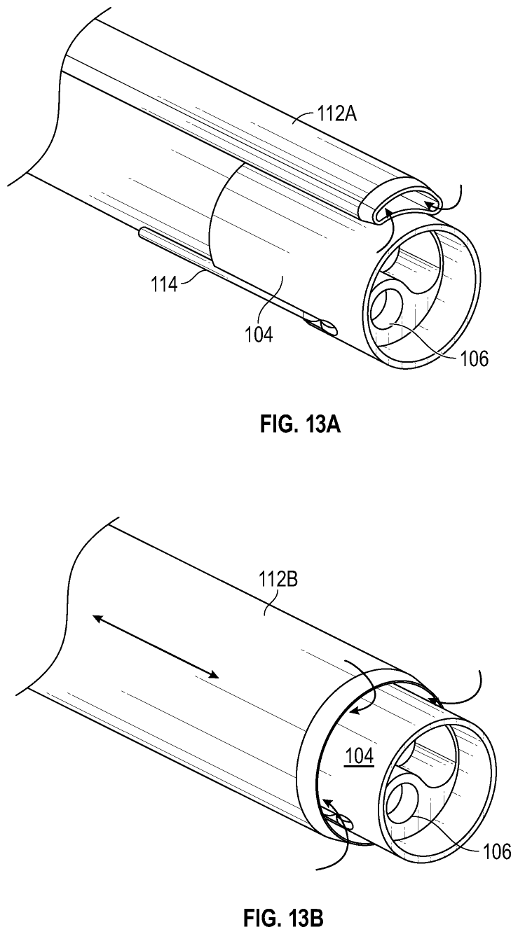

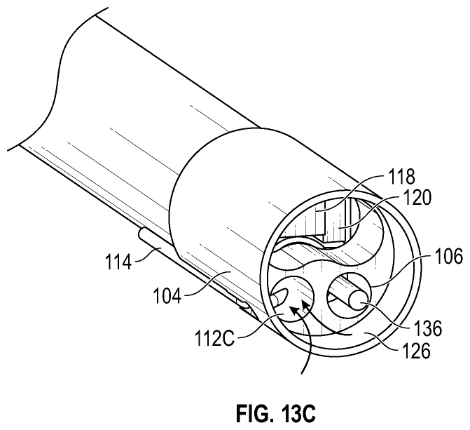

FIGS. 13A-13C illustrate variations of a secondary lumen 112. The secondary lumen 112 can be an integrated outflow lumen. The secondary lumen 112 can be and/or include a dedicated lumen configured to communicate with the location internal to the subject. The secondary lumen 112 can be used to ensure IRP remains in acceptable ranges and/or for the evacuation of biological objects. The secondary lumen 112 can be connected to various means for controlling IRP and fluid flow as described herein with reference to FIGS. 3A-3F. The secondary lumen 112 can be an advanced integrated channel/sheath designed to eliminate the need for a traditional ureteral access sheath.

As shown in FIG. 13A, the ureteroscope 100 can include a secondary lumen 112A. The secondary lumen 112A can be an integrated outer tube. The secondary lumen 112A can extend along a portion of the outer surface of the ureteroscope 100. Fluid can be configured to flow into the secondary lumen 112A at the distal end of the ureteroscope 100. As shown in FIG. 13A, the secondary lumen 11A can be integrated externally along the shaft's outer diameter, optimizing fluid outflow from the intrarenal space. In some examples, the secondary lumen 112A can include a hydrophilic coating for facilitating an easier insertion of the ureteroscope 100 and reduce trauma to the subject.

As shown in FIG. 13B, the ureteroscope 100 can include a secondary lumen 112B. The secondary lumen 112B can be an integrated outer tube. The secondary lumen 112A can extend around the outer surface of the ureteroscope 100. Fluid can be configured to flow into the secondary lumen 112A at the distal end of the ureteroscope 100. The secondary lumen 112B can be coaxial channel along the distal section of a ureteroscope 100. The secondary lumen 112B can create an annular fluid path between the ureteroscope and the outer sheath. This sheath can utilize the ureteroscope's deflection capabilities for precise navigation and may telescope axially relative to the ureteroscope 100, enabling highly accurate positioning for effective stone evacuation. In some examples, the secondary lumen 112B can form a cup for the distal tip. For example, the secondary lumen 112B may extend distally beyond the distal end of the ureteroscope 100. In such examples, the distal tip 104 may not include the cup 126 and can rely on the secondary lumen 112B. For example, the secondary lumen 112B may be positioned around the ureteroscope shown in FIGS. 10A-10B. In some examples, the secondary lumen 112A can include a hydrophilic coating for facilitating an easier insertion of the ureteroscope 100 and reduce trauma to the subject.

As shown in FIG. 13C, the ureteroscope 100 can include a secondary lumen 112C. The secondary lumen 112C can be integrated within the ureteroscope 100. For example, the secondary lumen 112C can extend in parallel with the evacuation lumen 116 within the ureteroscope 100. As shown in FIG. 13C, the secondary lumen 112C can be integrated either within the ureteroscope shaft alongside the working channel, optimizing fluid outflow from the intrarenal space.

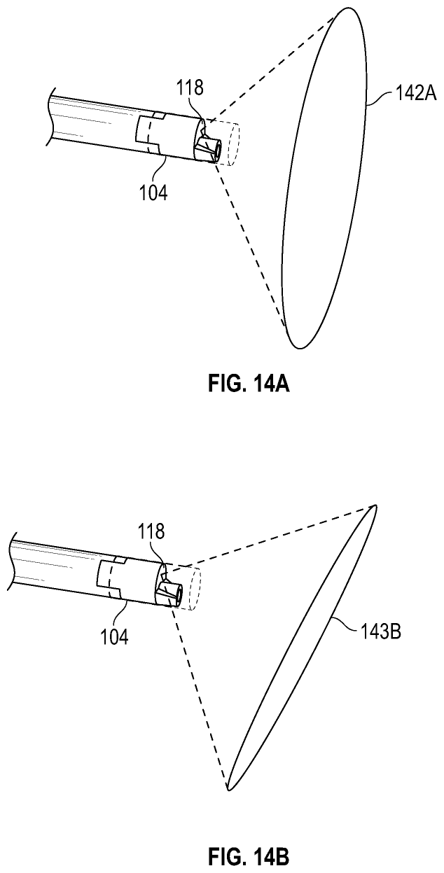

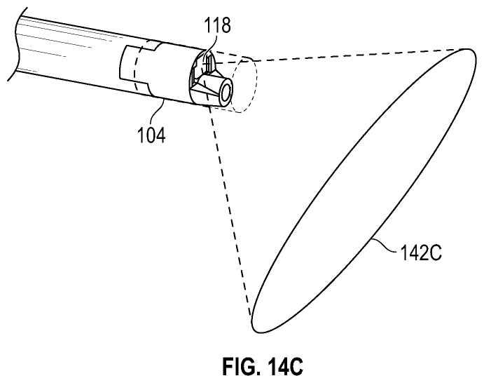

FIGS. 14A-14C illustrate various fields of view (FOV) and angles of view (AOV). Each FOV can be conical extending equally about a central axis.

FIG. 14A illustrates a first FOV 142A with a first AOV. The first AOV can be 0 or about 0 degrees such that the central axis of the first FOV 142A extends longitudinally from the optical sensor 118.

FIG. 14B illustrates a second FOV 142B with a second AOV. The second AOV can be an acute angle such that the central axis of the second FOV 142B extends at an acute angle from the longitudinal axis of the optical sensor 118.

FIG. 14C illustrates a third FOV 142C with a third AOV. The third AOV can be an obtuse angle such that the central axis of the third FOV 142C extends at an obtuse angle from the longitudinal axis of the optical sensor 118.

The optical sensor 118 can be provided at an optimized angle for enhancing imaging. In some examples, the optimized angle can be precision-tilted to an acute angle between 10 or about 10 and 45 or about 45 degrees. The acute angle can be directed downward toward the ablation device 136. In some examples, the angle can provide optical ray-tracing and/or simulation optimization. The angle can further ensure maximum visibility of the ablation device 136 and improve accuracy of activating the ablation device 136.

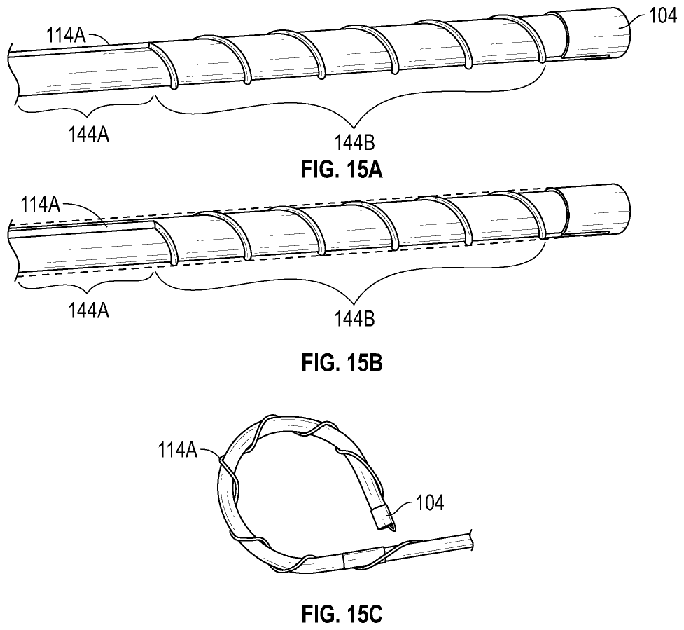

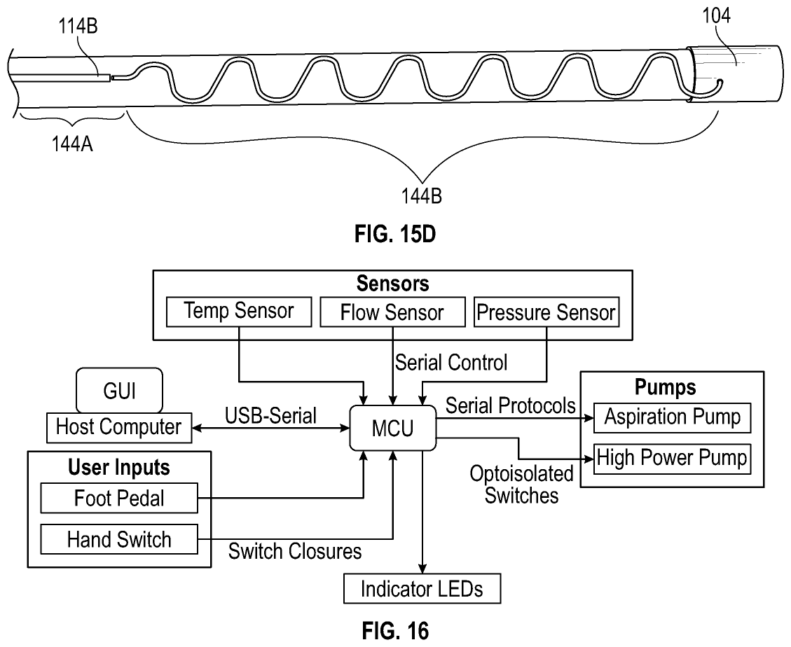

FIGS. 15A-15D illustrate various examples of a supply lumen 114. In some examples, the supply lumen 114 can be parallel with the longitudinal axis of the ureteroscope 100. As shown in FIGS. 15A-15C, the supply lumen 114 can extend in a helical, zigzag, and/or sinusoidal configuration relative to the longitudinal axis of the ureteroscope 100. The ureteroscope 100 can be navigated to a target site. To assist the navigation, the ureteroscope 100 can include a non-articulating section 144A and an articulating section 144B. The articulating section 144B can be configured to be driven by a mechanism (e.g., pull wires) to bend and accommodate the anatomical structures of the subject. The helical, zigzag, and/or sinusoidal configurations can assist the supply lumen 114 in traversing an articulating section 144B of the ureteroscope 100 while maintaining flexibility.

FIGS. 15A-15C illustrate a helical supply lumen 114A extending around the ureteroscope 100. In some examples, the helical portion of the helical supply lumen 114A may be in the articulating section 144B. For example, the helical supply lumen 114A may include a linear portion extending along the non-articulating section 144A and a helical section extending along the articulating section 144B. The helical supply lumen 114A is flexible in bending and can stay in place around the ureteroscope 100. Additionally, the helical supply lumen 114A can be easily manufactured. In some examples, the helical supply lumen 114A can be integrated outside of the ureteroscope 100 as shown in FIG. 15A. In some examples, the helical supply lumen 114A can be integrated inside the ureteroscope 100 as shown in FIG. 15B.

FIG. 15C illustrates a sinusoidal supply lumen 114B extending along the ureteroscope 100. The sinusoidal supply lumen 114B is flexible in bending along a bending plane. In some examples, the diameter of the sinusoidal supply lumen 114B can be less than the helical supply lumen 114A.

Additionally and/or alternatively, the supply lumen 114 can include advanced flexibility technologies. For example, the supply lumen 114 can include a super elastic alloy. In some examples, the super elastic alloy can be a super elastic Nitinol and shape memory alloy. The alloy can enhance flexibility and ensure precise articulation without fatigue. In some examples, the supply lumen 114 can include a mirco-textured surface and/or lubricious coating. The micro-textured surface and/or coating can reduce friction and improve durability.

FIG. 16 illustrates a hardware block diagram illustrating controlling a ureteroscope, such as the ureteroscope 100 using electronic control circuitry, such as a microcontroller. The hardware can include computer devices, user input devices, pumps, sensors, and other devices. The computer devices can include a host computer providing a graphical user interface (GUI). The user inputs can include actuatable devices. For example, the user inputs can include a foot pedal and/or a hand switch. The pumps can include an aspiration pump and a high-power pump (e.g., the aspiration pump and the irrigation pump described herein with reference to FIGS. 3A-3B). The sensors can include a temperature sensor, a flow sensor, and/or a pressure sensor. In some examples, the sensor can further include an optical sensor. For example, the optical sensor 118. Each of the computer devices, user input devices, pumps, sensors, and other devices can be electrically coupled to the control circuitry. For example, the computer devices can be coupled to the control circuitry via a USB-serial connection. The user inputs can be coupled to the control circuitry via switches. The pumps can be coupled to the control circuitry via serial control and/or optoisolated switches. The sensors can be coupled to the control circuitry via serial protocols. In some examples, the computer devices, the user inputs, and the sensors can provide inputs into the control circuitry. In some examples, the pumps and other devices (e.g., indicators, such as LEDs) can be configured to receive an output from the control circuitry.

In some examples, the sensors can be integrated and/or miniature pressure and temperature sensors. Miniaturized pressure and temperature sensors can be embedded at the distal tip of the ureteroscope. For example, the pressure and temperature sensors can be positioned near the light sources 120 and optical sensor 118. Additionally and/or alternatively, the pressure and temperature sensors can be embedded at the proximal end of the ureteroscope. Methods to integrate the pressure and temperature sensors can include MEMS Pressure Sensors & Thin-Film Thermocouples and/or wireless telemetry. MEMS Pressure Sensors and Thin-Film Thermocouples can be directly embedded within the distal tip of the ureteroscope 100 for enhanced accuracy and reliability. Wireless telemetry can enable real-time continuous data transmission and monitoring. The sensors can include automated alarms and feedback controls to dynamically regulate intrarenal pressure and temperature within safe thresholds.

The sensors can provide advanced flow and pressure sensing. The sensors can be integrated within the ureteroscope and configured to enhance performance and provide real-time feedback. For example, along an inflow path, the sensors can include an ultrasonic doppler flow sensor and/or MEMS pressure sensor. The sensors can thereby monitor the inflow path without contaminating the fluid. Along the outflow path, high-precision, non-contact ultrasonic flow and pressure sensors can evaluate eductor pump effectiveness. Along the passive outflow path, dual flow and pressure sensors can be configured to detect subtle changes in fluid dynamics indicative of an obstruction.

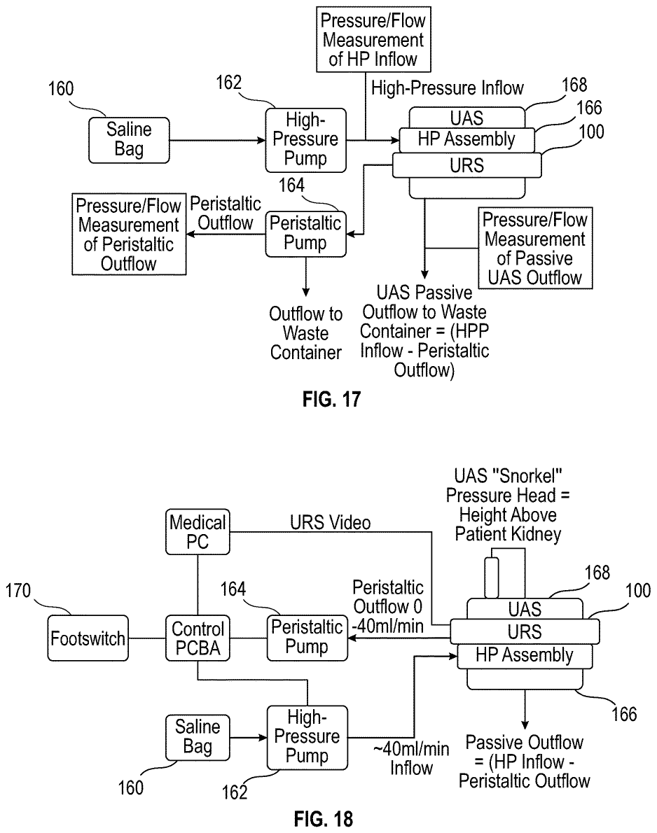

FIG. 17 illustrates a fluid flow block diagram for directing fluid with a ureteroscope, such as the ureteroscope 100. The illustrated components can include the ureteroscope 100, a fluid source 160 such as a saline bag, a high-pressure pump 162 (irrigation pump) coupled to the fluid source 160 and a supply lumen 114 of the ureteroscope 100, and an aspiration pump (such as, a peristaltic pump 164) coupled to an evacuation lumen 116 of the ureteroscope 100. An inflow and/or inlet pressure can be measured distal of the high-pressure pump 162. An outflow and/or outlet pressure can be measured distal of the peristaltic pump 164. Additionally, a passive outflow can pass through the ureteroscope 100 uncoupled to either the high-pressure pump 162 or the peristaltic pump 164. The passive outflow and/or pressure of the passive outflow can be measured distal of the ureteroscope 100. In some examples, a high pressure assembly 166 and a urethral access sheath 168 may be used with the ureteroscope 100.

FIG. 18 illustrates a functional block diagram for operating a ureteroscope, such as the ureteroscope 100. The functional block diagram can include aspects of the hardware block diagram and the fluid flow block diagram described herein with reference to FIGS. 16-17. For example, the functional block diagram can include computer devices (also referred to as a medical PC), electronic control circuitry (illustrated as Control PCBA), pumps (e.g., a high-pressure pump 162 and/or a peristaltic pump 164/aspiration pump), user input devices 170 (e.g., a footswitch), and a fluid source 160 (e.g., saline bag). The user input devices, pumps, and computer devices can be electrically connected to the control circuitry. The computer devices can be further electrically coupled to the ureteroscope 100. For example, the computer devices can receive video from an optical sensor 118 of the ureteroscope 100. The high-pressure pump 162 can be coupled to the fluid source 160 and a supply lumen 114 of the ureteroscope 100. The high-pressure pump 162 can be controlled to input a fluid flow into the supply lumen 114 of the ureteroscope 100. For example, the high-pressure pump 162 can provide a fluid flow rate of 40 or about 40 ml/min. The peristaltic pump 164 can be coupled to the evacuation lumen 116 of the ureteroscope 100. The peristaltic pump 164 can be controlled to regulate a fluid outflow from the evacuation lumen of the ureteroscope 100. For example, the peristaltic pump 164 can regulate the outflow between 0 or about 0 and 40 or about 40 ml/min. In some examples, the ureteroscope 100 can include a snorkel for passive outflow.

The control circuitry can be configured to manage the flow within the ureteroscope. In some examples, the control circuitry can be configured to clear clogs and blockages within the ureteroscope 100. For example, the control circuitry can be configured to automatically reverse flow to proactively clear clogs and release undesired fragments or tissues. In some examples, the control circuitry can execute one or more advanced processes configured to detect flow anomalies and initiate automated, controlled flow reversal sequences. In some examples, the flow reversal sequence can be initiated by a user input device such as a footswitch, a handle-mounted button, and/or voice control.

As described herein with reference to FIGS. 16 and 18, the computer devices can be electrically coupled to the ureteroscope 100. In particular, the computer devices can be electrically coupled to the optical sensor 118. In some examples, the control circuitry can implement a comprehensive artificial intelligence (AI) and/or computer vision. AI-driven features can enhance efficiency, safety, and procedural outcomes. For example, AI and/or computer vision can be sued to identify differences between tissue and stones and can be used to dynamically control suction and ablation outputs. In some examples, the AI can adjust the amplitudes and frequencies of the suction and ablation outputs based on real-time feedback from flow and pressure sensors. In some examples, the AI and computer vision can identify the composition of a biological object and automatically optimize ablation energy, frequency, and suction parameters. In some examples, the AI can predict flow rates and pressure adjustments by anticipating surgical conditions and adjusting the suction and ablation outputs accordingly. In some examples, the AI can detect cavity collapse and compensate for the intrarenal pressure. In some examples, the AI can monitor the wear and longevity of the components of the ureteroscope. For example, the AI can provide a real-time assessment of component longevity and preemptive maintenance recommendations. In some examples, the AI can dynamically adjust suction and versal patterns based on obstruction analysis to detect and clear clogs. In some examples, the AI can improve visualization of the biological objects and anatomical structures by enhancing contrast in real-time. In some examples, the AI can enhance procedural guidance and risk mitigation by providing anatomical mapping.

Additionally, a robotic system can automate essential ureteroscopic functions. In some examples, the robotic system can automate rotation, deflection, insertion, and withdrawal, with real-time positional feedback. In some examples, computer vision can be implemented with robotics to semi-automate or fully automate the entire stone removal procedure.

The following describes a method for an automated workflow. The method begins with a step of initially inserting and positioning the ureteroscope within a subject. The ureteroscope can be manually inserted into the subject. An AI-powered robotic system can assist in navigating the distal tip of the ureteroscope to the middle of the target cavity/anatomy using computer vision and real-time tracking algorithms. The AI-powered robotic system can also identify anatomical landmarks and highly potential stone locations using an AI-enhanced mapping technology.

The method then moves to a step of robotically controlling integration by attaching the ureteroscope to a robotic receptacle. The robotic receptacle can precisely manipulate the overall movement of the ureteroscope. The AI-powered robotic system can interpret real-time imaging and sensor feedback to assist or autonomously guide movement of the ureteroscope.

The method then moves to a step of autonomously navigating the ureteroscope towards a biological object. The AI-powered robotic system can use computer vision and an AI-enhanced trajectory planning system to navigate towards the biological object. In some examples, the AI-powered robotic system can dynamically adjust angulation, approach vectors, and fluid dynamics for optimal stone access.

The method then moves to a step of fragmenting and capturing biological objects. Once aligned with a biological object, the AI-powered robotic system can automatically engage venturi or entrainment-assisted suction to attract the biological object towards the distal tip of the ureteroscope. The AI-powered robotic system can then analyze the size, composition, and fragility of the biological object to optimize ablation parameters for efficient fragmentation while minimizing thermal damage to surrounding tissues. The AI-powered robotic system can dynamically adjust the position, energy output, and pulse frequency of the ablation device based on real-time responses of the biological object.

The method then moves to a step of retrieving the biological object. After fragmenting the biological object, the AI-powered robotic system can optionally retrieve the fragments using venturi or entrainment-assisted suction as a grasping mechanism rather than breaking the biological object to dust. This step can include retracting the ureteroscope with the fragmented biological object drawn to the distal end of the ureteroscope via venturi and/or entrainment assisted suction.

The method then moves to a step of completing and extracting the ureteroscope. Upon completion of the procedure (i.e., evacuating the location internal to the subject of biological objects), the AI-powered robotic system can signal to the user that the procedure is complete. The user can manually remove the ureteroscope. Alternatively, the AI-powered robotic system can active a retraction mode to retract the ureteroscope itself. In some examples, AI can verify whether the location internal to the subject is free of biological objects before concluding the procedure.

### Example Implementations

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any one or more features of one of the example clauses listed below can be combined by any of the one or more features of any one or more other example clauses listed below or any of the features described herein.

Clause 1. A system for removing a biological object from an anatomical structure, the system comprising: an elongated tubular body comprising a proximal end and a distal end configured to be inserted into the anatomical structure, the elongated tubular body further comprising one or more aspiration openings configured to receive the biological object; a supply lumen configured to provide a fluid flow into the elongated tubular body; an evacuation lumen configured to evacuate the fluid flow and the biological object from the anatomical structure; and a distal tip positioned at the distal end of the elongated tubular body, the distal tip comprising an optically transparent material.

Clause 2. The system of Clause 1, wherein the distal tip further comprises a cup, a choke, and a working channel extension.

Clause 3. The system of Clause 2, wherein the cup comprises an annular wall extending distally away from the distal end of the elongated tubular body.

Clause 4. The system of any one of Clauses 2-3, wherein the cup is formed of a polymer.

Clause 5. The system of any one of Clauses 2-3, wherein the cup is formed of a jewel.

Clause 6. The system of any one of Clauses 2-5, wherein the cup has a length between about 0.1 mm and about 2.0 mm.

Clause 7. The system of any one of Clauses 2-6, wherein the choke comprises a constricted lumen.

Clause 8. The system of any one of Clauses 2-7, wherein the choke is configured to be thermal resistant and laser resistant.

Clause 9. The system of any one of Clauses 2-8, wherein the choke is formed of a jewel.

Clause 10. The system of any one of Clauses 2-9, wherein the choke has a length of about 0.1 mm to about 1.0 mm.

Clause 11. The system of any one of Clauses 1-10, wherein the supply lumen is configured to provide the fluid flow into the elongated tubular body as a liquid jet.

Clause 12. The system of any one of Clauses 1-10, wherein the supply lumen is configured to induce a Venturi-assisted suction at the one or more aspiration openings.

Clause 13. The system of any one of Clauses 1-10, wherein the supply lumen is configured to induce an Entrainment-assisted suction at the one or more aspiration openings.

Clause 14. The system of any one of Clauses 1-13, wherein the supply lumen extends distally along the elongated tubular body and comprises a bend configured to redirect the fluid flow in a proximal direction.

Clause 15. The system of any one of Clauses 1-13, wherein the supply lumen comprises a J-bend.

Clause 16. The system of any one of Clauses 1-15, wherein the supply lumen comprises an incidence angle between about 10 degrees and about 60 degrees.

Clause 17. The system of any one of Clauses 1-16, wherein the supply lumen is positioned at a radial distance from an inner surface of the evacuation lumen.

Clause 18. The system of Clause 17, wherein the radial distance is between about 0.0 mm and about 0.5 mm.

Clause 19. The system of any one of Clauses 1-18, wherein the elongated tubular body further comprises one or more light sources and an image sensor.

Clause 20. The system of any one of Clauses 1-19, wherein the one or more aspiration openings are positioned at a distal face of the elongated tubular body.

Clause 21. The system of any one of Clauses 1-20, wherein the elongated tubular body further comprises one or more vent openings.

Clause 22. The system of Clause 21, wherein the one or more vent openings are offset from the distal end of the elongated tubular body by a distance of about 2.0 mm to about 10.0 mm.

Clause 23. The system of any one of Clauses 21-22, wherein the one or more vent openings are circular.

Clause 24. The system of any one of Clauses 21-22, wherein the one or more vent openings are longitudinal slots.

Clause 25. The system of any one of Clauses 21-22, wherein the one or more vent openings are slots arranged orthogonally to the longitudinal axis of the elongated tubular body.

Clause 26. The system of any one of Clauses 2-25, wherein the choke is clover shaped.

Clause 27. The system of any one of Clauses 2-25, wherein the choke is circular shaped.

Clause 28. The system of any one of Clauses 2-25, wherein the choke comprises a plurality of slots.

Clause 29. The system of any one of Clauses 1-28, wherein the elongated tubular body is configured to receive an ablation device.

Clause 30. The system of Clause 29, wherein the ablation device is an ultrasonic device.

Clause 31. The system of Clause 29, wherein the ablation device is a laser device.

Clause 32. The system of any one of Clauses 1-31, further comprising a secondary lumen.

Clause 33. The system of any one of Clauses 29-32, further comprising a reciprocation mechanism.

Clause 34. The system of Clause 33, wherein the reciprocation mechanism is configured to move the ablation device longitudinally.

Clause 35. The system of Clause 34, wherein the reciprocation mechanism automated is configured to move the ablation device between about 0.1 mm and about 2.0 mm.

Clause 36. The system of any one of the preceding clauses, wherein the system comprises a ureteroscope configured to be inserted into a urinary tract and the biological object comprises a kidney stone.

Clause 37. A system for removing a biological object from a location internal to a subject, the system comprising: a supply lumen configured to receive a first fluid flow and to redirect the first fluid flow from a first direction to a second direction; an evacuation lumen configured to receive the first fluid flow and a second fluid flow travelling in the second direction from the location internal to the subject toward the evacuation lumen, the second fluid flow carrying the biological object from the location internal to the subject toward the evacuation lumen; an aspiration opening in fluid communication with the evacuation lumen, wherein the aspiration opening is configured to receive the second fluid flow from the location internal to the subject as a result of the first fluid flow entering the evacuation lumen; and a distal tip extending distally from a distal end of the evacuation lumen, the distal tip comprising a working channel extension extending from a distal end of the distal tip to a proximal end of the distal tip, wherein the working channel extension is in fluid communication with the evacuation lumen, wherein the distal tip is configured to enhance visibility for an optical sensor configured to provide images of the location internal to the subject and to prevent the biological object from clogging the evacuation lumen.

Clause 38. The system of Clause 37, wherein the distal tip comprises a choke positioned in the working channel extension and defining one or more constrictions at one or more axial positions between the distal end and the proximal end of the distal tip, and wherein the choke has an effective cross-sectional area smaller than a cross-sectional area of the evacuation lumen.

Clause 39. The system of any one of Clauses 37-38, wherein the distal tip further comprises a cup having an annular wall extending distally from the distal end of the evacuation lumen, and wherein the cup is configured to retain the biological object near the aspiration opening.

Clause 40. The system of any one of Clauses 37-39, wherein the distal tip is formed from a laser energy resistant material.

Clause 41. The system of any one of Clauses 37-40, wherein the distal tip is formed from an optically transparent material.

Clause 42. The system of any one of Clauses 37-41, wherein the evacuation lumen and the distal tip are configured to receive an ablation device.

Clause 43. The system of Clause 42, further comprising a mechanism configured to move the ablation device in the evacuation lumen.

Clause 44. A system for removing a biological object from a location internal to a subject, the system comprising: an elongated tubular body comprising: a supply lumen extending distally from a proximal end of the elongated tubular body, wherein the supply lumen is configured to be in fluid communication with a fluid source and configured to redirect a first fluid flow from the fluid source; and an evacuation lumen having a first cross-sectional area extending from a distal end of the elongated tubular body to the proximal end of the elongated tubular body, wherein the evacuation lumen is configured to receive the first fluid flow from the supply lumen after being redirected into the evacuation lumen; and a distal tip extending distally from a distal end of the elongated tubular body, the distal tip comprising: a working channel extension in fluid communication with the evacuation lumen, wherein a distal end of the working channel extension defines an aspiration opening configured to receive a second fluid flow as a result of the first fluid flow entering the evacuation lumen; and a choke having a second cross-sectional area disposed within the working channel extension, wherein the second cross-sectional area is smaller than the first cross-sectional area.

Clause 45. The system of Clause 44, wherein the choke is selected from the group consisting of a three clover shape, a four clover shape, a plurality of slots, a plurality of aspiration openings, and two hemispheric aspiration openings.

Clause 46. The system of any one of Clauses 44-45, wherein the distal tip further comprises a cup, and wherein: the choke has an axial length between about 0.1 mm to about 0.5 mm; and the cup has an axial length between about 0.1 mm and about 2.0 mm.

Clause 47. The system of any one of Clauses 44-46, wherein the distal tip is formed from a jewel.

Clause 48. The system of any one of Clauses 44-47, further comprising an ablation device extending through the evacuation lumen and the distal tip.

Clause 49. The system of any one of Clauses 44-48, wherein the evacuation lumen is configured to receive an ablation device, and wherein the system further comprises a reciprocating mechanism configured to reciprocate the ablation device in the evacuation lumen.

Clause 50. The system of Clause 49, wherein the choke supports the ablation device.

Clause 51. A system for removing a biological object from a location internal to a subject, the system comprising: an elongated tubular structure comprising a working channel having a first effective cross-sectional area; and a distal tip extending distally from a distal end of the elongated tubular structure, the distal tip comprising: a working channel extension extending from a distal end of the distal tip to a proximal end of the distal tip, wherein the working channel extension is in fluid communication with the working channel; and a choke positioned within the working channel extension at an axial location between a distal end of the working channel extension and a proximal end of the working channel extension, wherein the choke has a second effective cross-sectional area smaller than the first effective cross-sectional area; wherein the system is configured to aspirate the biological object through the choke, the working channel extension, and the working channel.

Clause 52. The system of Clause 51, wherein the choke is clover shaped having four fingers extending radially inward from the distal tip.

Clause 53. The system of any one of Clauses 51-52, wherein the choke is clover shaped having three fingers extending radially inward from the distal tip.

Clause 54. The system of any one of Clauses 51-53, wherein the choke comprises a plurality of slots.

Clause 55. The system of any one of Clauses 51-54, wherein the choke comprises a plurality of aspiration openings.

Clause 56. The system of any one of Clauses 51-55, wherein the choke comprises two hemispheric openings.

Clause 57. The system of any one of Clauses 51-56, wherein the elongated tubular structure further comprises an optical sensor positioned along a first axis parallel to a central axis of the working channel and a distal end of the working channel extension is axially displaced from a distal end of the optical sensor by a distance.

Clause 58. The system of Clause 57, further comprising an ablation device extending through the working channel and the working channel extension, wherein a distal end of the ablation device is axially aligned with the distal end of the working channel extension.

Clause 59. The system of any one of Clauses 57-58, wherein the distance is a first distance, wherein the distal end of the distal tip is axially displaced from the optical sensor by a second distance.

Clause 60. The system of Clause 59, wherein the first distance maintains a separation between a distal end of an ablation device and the optical sensor and the second distance maintains a separation between an aspiration opening and the optical sensor.

Clause 61. The system of any one of Clauses 51-60, wherein the distal tip further comprises a cup, wherein the cup extends distally from a distal end of the working channel extension.

Clause 62. A system for removing a biological object from a location internal to a subject, the system comprising: an elongated tubular body comprising: an optical sensor; a supply lumen configured to be in fluid communication with a fluid source; and an evacuation lumen in fluid communication with the supply lumen; a distal tip extending distally from a distal end of the elongated tubular body, the distal tip comprising: a working channel extension in fluid communication with the evacuation lumen, wherein a distal end of the working channel extension defines an aspiration opening configured to be in fluid communication with the location internal to the subject; and a choke disposed within the working channel extension; an ablation device extending through the evacuation lumen and the distal tip; and a controller configured to receive optical signals from the optical sensor and differentiate between tissue and the biological object.

Clause 63. The system of Clause 62, wherein the supply lumen introduces a liquid jet with fluid from the fluid source into the evacuation lumen to create a vacuum within the working channel extension, and wherein the controller is configured to automatically control a level of the vacuum based at least partially on a signal from a flow sensor.

Clause 64. The system of any one of Clauses 62-63, wherein the controller is configured to automatically control the ablation device based at least partially on a differentiation between a tissue and the biological object.

Clause 65. The system of any one of Clauses 62-64, further comprising a reciprocating mechanism coupled to the ablation device, wherein the controller is configured to automatically control, based at least partially on the optical signals, the reciprocating mechanism to cause movement of the ablation device relative to the elongated tubular body.

Clause 66. A system for removing a biological object from a location internal to a subject, the system comprising: an irrigation pump configured to introduce a first fluid flow into a supply lumen; an aspiration pump; an elongate tubular body comprising: an evacuation lumen extending between a distal opening at a distal end and a proximal opening at a proximal end, wherein the evacuation lumen is in fluid communication with the supply lumen adjacent to the distal opening and is in fluid communication with the aspiration pump at the proximal opening; the supply lumen in fluid communication with the irrigation pump, the supply lumen configured to introduce the first fluid flow as a liquid jet into the evacuation lumen to create a first vacuum level at the distal opening to entrain a second fluid flow from the location internal to the subject into the evacuation lumen through the distal opening and as a result remove the biological object from the location internal to the subject; and one or more vent openings disposed within the evacuation lumen at the distal end; one or more sensors; and a controller in communication with the irrigation pump, the aspiration pump, and the one or more sensors; wherein: the aspiration pump is configured to create a second vacuum level at the proximal opening; and the controller is configured to manage pressures and temperatures within the location internal to the subject.

Clause 67. The system of Clause 66, wherein the controller is configured to simultaneously control the irrigation pump and the aspiration pump.

Clause 68. The system of any one of Clauses 66-67, wherein the one or more vent openings are configured to recirculate at least a portion of the second fluid flow from the evacuation lumen into the location internal to the subject.

Clause 69. The system of any one of Clauses 66-68, wherein the one or more vent port openings comprise a coating configured to prevent the biological object from adhering to the evacuation lumen.

Clause 70. The system of any one of Clauses 66-69, wherein the elongate tubular body further comprises an imaging device.

Clause 71. The system of Clause 70, wherein the controller is configured to control the irrigation pump and the aspiration pump in response to signals generated by the one or more sensors or the imaging device.

Clause 72. The system of any one of Clauses 66-71, further comprising a secondary lumen movably disposed along an external surface of the elongate tubular body, wherein the secondary lumen is configured to aspirate a third fluid flow.

Clause 73. A system for removing a biological object from a location internal to a subject, the system comprising: an elongate tubular body comprising: a supply lumen configured to be in fluid communication with an irrigation pump configured to provide a first fluid flow through the supply lumen; an evacuation lumen extending between a distal opening and a proximal opening, wherein the evacuation lumen is in fluid communication with the supply lumen adjacent to the distal opening and is configured to be in fluid communication with an aspiration pump at the proximal opening; and one or more vent openings disposed along the evacuation lumen; and a controller in communication with the irrigation pump, the aspiration pump, and one or more sensors.

Clause 74. The system of Clause 73, wherein the one or more vent openings are positioned proximally from the distal opening.

Clause 75. The system of any one of Clauses 73-74, wherein the one or more vent openings are selected from the group consisting of a single circular opening, a single longitudinal slot, and multiple orthogonal slots.

Clause 76. The system of any one of Clauses 73-75, wherein the one or more vent openings comprise a biocompatible hydrophobic coating.

Clause 77. The system of any one of Clauses 73-76, wherein the supply lumen redirects the first fluid flow into the evacuation lumen as a liquid jet to create a first vacuum level for entraining a second fluid flow from the location internal to the subject into the evacuation lumen thereby aspirating the biological object.

Clause 78. The system of any one of Clauses 73-77, wherein supply lumen redirects a first fluid flow at an angle between 35 degrees and 55 degrees relative to a longitudinal axis of the evacuation lumen.

Clause 79. The system of any one of Clauses 73-78, further comprising a secondary lumen movably disposed along an external surface of the elongate tubular body.

Clause 80. The system of any one of Clauses 73-79, wherein a portion of the supply lumen extends in a helical, zigzag, or sinusoidal configuration relative to a longitudinal axis of the elongate tubular body, wherein the portion of the supply lumen is located in a zone of articulation of the elongate tubular body.

Clause 81. A method for removing a biological object from a location internal to a subject, the method comprising: pumping a first fluid flow through a supply lumen; redirecting the first fluid flow into an evacuation lumen to create a vacuum for entraining a second fluid flow from the location internal to the subject into the evacuation lumen and thereby aspirating the biological object through the evacuation lumen; recirculating at least a portion of the second fluid flow through one or more vent openings from the evacuation lumen into the location internal to the subject; and aspirating the first fluid flow and the second fluid flow through the evacuation lumen.

Clause 82. The method of Clause 81, wherein creating the vacuum comprises creating a first vacuum level at a distal end of the evacuation lumen, wherein aspirating the first fluid flow and the second fluid flow through the evacuation lumen comprises creating a second vacuum level at a proximal end of the evacuation lumen, and wherein the first vacuum level and the second vacuum level in combination act to aspirate the first fluid flow and the second fluid flow through the evacuation lumen.

Clause 83. The method of any one of Clauses 81-82, further comprising controlling an irrigation pump to control the first vacuum level and controlling an aspiration pump to control the second vacuum level.

Clause 84. The method of Clause 83, wherein controlling the irrigation pump and the aspiration pump is based at least in part on signals from one or more sensors.

Clause 85. The method of any one of Clauses 81-84, wherein recirculating at least the portion of the second fluid flow through the one or more vent openings from the evacuation lumen into the location internal to the subject regulates a pressure and a temperature within the location internal to the subject.

Clause 86. The method of any one of Clauses 81-85, wherein recirculating at least the portion of the second fluid flow through the one or more vent openings from the evacuation lumen into the location internal to the subject is based on a relationship among the distal opening, a direction of the first fluid flow due to redirecting the first fluid flow, and the one or more vent openings to increase the second fluid flow relative to the first fluid flow.

Clause 87. A system for facilitating removal of a biological object from a location internal to a subject, the system comprising: an elongated tubular structure comprising a working channel having a first effective cross-sectional area; and an optical sensor positioned adjacent to the working channel; and a distal tip projecting from a distal end of the elongated tubular structure, the distal tip comprising: a working channel extension defined through the distal tip and in fluid communication with the working channel; and a restriction defined within the working channel extension and having a second effective cross-sectional area which is less than the first effective cross-sectional area, wherein the optical sensor is positioned proximally of a distal end of the working channel extension by a first distance and is further positioned proximally of the restriction by a second distance which is less than the first distance.

Clause 88. The system of Clause 87, wherein the restriction is clover shaped having four fingers extending radially inward from the distal tip.

Clause 89. The system of any one of Clauses 87-88, wherein the restriction is clover shaped having three fingers extending radially inward from the distal tip.

Clause 90. The system of any one of Clauses 87-89, wherein the restriction comprises a plurality of slots.

Clause 91. The system of any one of Clauses 87-90, wherein the restriction comprises a plurality of aspiration openings.

Clause 92. The system of any one of Clauses 87-91, wherein the restriction comprises two hemispheric openings.

Clause 93. The system of any one of Clauses 87-92, wherein the optical sensor is positioned along a first axis parallel to a central axis of the working channel and the distal end of the working channel extension is axially displaced from a distal end of the optical sensor.

Clause 94. The system of Clause 93, further comprising an ablation device extending through the working channel and the working channel extension, wherein a distal end of the ablation device is axially aligned with the distal end of the working channel extension.

Clause 95. The system of any one of Clauses 93-94, wherein distal end of the distal tip is axially displaced from the optical sensor by the second distance.

Clause 96. The system of any one of Clauses 87-95, wherein the first distance maintains a separation between a distal end of an ablation device and the optical sensor and the second distance maintains a separation between an aspiration opening and the optical sensor.

Clause 97. The system of any one of Clauses 87-96, wherein the distal tip further comprises a cup, wherein the cup extends distally from the distal end of the working channel extension.

Clause 98. A method of operating the system of any one of the preceding clauses.

Clause 99. A system, device, and/or method as illustrated and/or described.

### Other Variations

Any of the implementations disclosed herein can include one or more features disclosed in one or more of U.S. Pat. No. 12,419,656 or U.S. patent application Ser. No. 19/373596, titled SYSTEMS AND METHODS FOR REMOVAL OF BIOLOGICAL OBJECT FROM ANATOMICAL STRUCTURES WITHIN A BODY filed on Oct. 29, 2025, each of which is incorporated by reference in its entirety.

While certain examples have been described in the context of ureteroscopy, the approaches described herein can be used for any medical procedure that utilizes a catheter, such as any medical procedure directed to removing a biological object from an anatomical structure within the body. While certain examples have been described in the context of removing solid deposits, the approaches described herein can be used for removing any biological object, which may not necessarily be a solid.

The foregoing description details certain examples of the systems, devices, and methods disclosed herein. It will be appreciated, however, that no matter how detailed the foregoing appears in text, the systems, devices, and methods can be practiced in many ways. It should be noted that the use of particular terminology when describing certain features or aspects of the disclosure should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the examples. It will also be appreciated by those of skill in the art that parts included in one example are interchangeable with other examples; one or more parts from a depicted example can be included with other depicted examples in any combination. For example, any of the various components described herein and/or depicted in the Figures may be combined, interchanged or excluded from other examples.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Conditional language used herein, such as, among others, “can,” “could”, “might,” “may,” “e.g.,” and the like, unless specifically stated otherwise, or otherwise understood within the context as used, is generally intended to convey that certain implementation include, while other implementations do not include, certain features, elements and/or states. Thus, such conditional language is not generally intended to imply that features, elements and/or states are in any way required for one or more implementations or that one or more implementations necessarily include logic for deciding, with or without author input or prompting, whether these features, elements and/or states are included or are to be performed in any particular implementation. The term “each,” as used herein, in addition to having its ordinary meaning, can mean any subset of a set of elements to which the term “each” is applied.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated value.

It is noted that some examples above may be described as a process, which is depicted as a flowchart, a flow diagram, a structure diagram, or a block diagram. Although a flowchart may describe the operations as a sequential process, many of the operations can be performed in parallel, or concurrently, and the process can be repeated. In addition, the order of the operations may be rearranged. A process is terminated when its operations are completed. A process may correspond to a method, a function, a procedure, a subroutine, a subprogram, etc. When a process corresponds to a software function, its termination corresponds to a return of the function to the calling function or the main function.

Various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific examples disclosed above may be combined in different ways to form additional implementations, all of which fall within the scope of the present disclosure.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the examples disclosed herein. Consequently, it is not intended that this disclosure be limited to the specific examples disclosed herein, but that it covers all modifications and alternatives coming within the true scope and spirit of the disclosure as embodied in the attached claims.

1. A system for removing a biological object from a location internal to a subject, the system comprising:
an elongated tubular body comprising:
a supply lumen extending distally from a proximal end of the elongated tubular body, wherein the supply lumen is configured to be in fluid communication with a fluid source and configured to redirect a first fluid flow from the fluid source; and
an evacuation lumen having a first cross-sectional area extending from a distal end of the elongated tubular body to the proximal end of the elongated tubular body, the evacuation lumen comprising a working channel, wherein the evacuation lumen is configured to receive the first fluid flow from the supply lumen after being redirected into the evacuation lumen; and
a distal tip extending distally from a distal end of the elongated tubular body, the distal tip comprising:
a working channel extension in fluid communication with the working channel, wherein a distal end of the working channel extension defines an aspiration opening configured to receive a second fluid flow as a result of the first fluid flow entering the evacuation lumen; and
a choke having a second cross-sectional area disposed within the working channel extension, wherein the second cross-sectional area is smaller than the first cross-sectional area.

2. The system of claim 1, wherein the choke is selected from the group consisting of a three clover shape, a four clover shape, a plurality of slots, a plurality of aspiration openings, and two hemispheric aspiration openings.

3. The system of claim 1, wherein the distal tip further comprises a cup, and wherein:
the choke has an axial length between about 0.1 mm to about 0.5 mm; and
the cup has an axial length between about 0.1 mm and about 2.0 mm.

4. The system of claim 1, wherein the distal tip is formed from a jewel.

5. The system of claim 1, further comprising an ablation device extending through the working channel and the distal tip.

6. The system of claim 1, wherein the working channel is configured to receive an ablation device, and wherein the system further comprises a reciprocating mechanism configured to reciprocate the ablation device in the working channel.

7. The system of claim 5, wherein the choke supports the ablation device.

8. A system for removing a biological object from a location internal to a subject, the system comprising:
an elongated tubular body comprising:
an optical sensor;
a supply lumen configured to be in fluid communication with a fluid source; and
an evacuation lumen in fluid communication with the supply lumen, the evacuation lumen comprising a working channel;
a distal tip extending distally from a distal end of the elongated tubular body, the distal tip comprising:
a working channel extension in fluid communication with the working channel, wherein a distal end of the working channel extension defines an aspiration opening configured to be in fluid communication with the location internal to the subject; and
a choke disposed within the working channel extension;
an ablation device extending through the working channel and the distal tip; and
a controller configured to receive optical signals from the optical sensor and differentiate between tissue and the biological object.

9. The system of claim 8, wherein the supply lumen is configured to introduce a liquid jet with fluid from the fluid source into the evacuation lumen to create a vacuum within the working channel extension, and wherein the controller is configured to automatically control a level of the vacuum based at least partially on a signal from a flow sensor.

10. The system of claim 8, wherein the controller is configured to automatically control the ablation device based at least partially on a differentiation between tissue and the biological object.

11. The system of claim 8, further comprising a reciprocating mechanism configured to be coupled to the ablation device, wherein the controller is configured to automatically control, based at least partially on the optical signals, the reciprocating mechanism to cause movement of the ablation device relative to the elongated tubular body.

12. A system for facilitating removal of a biological object from a location internal to a subject, the system comprising:
an elongated tubular structure comprising a working channel having a first effective cross-sectional area;
an optical sensor positioned adjacent to the working channel; and
a distal tip projecting from a distal end of the elongated tubular structure, the distal tip comprising:
a working channel extension defined through the distal tip and in fluid communication with the working channel; and
a restriction defined within the working channel extension and having a second effective cross-sectional area which is less than the first effective cross-sectional area,
wherein the optical sensor is positioned proximally of a distal end of the working channel extension by a first distance and is further positioned proximally of the restriction by a second distance which is less than the first distance.

13. The system of claim 12, wherein the restriction is clover shaped having four fingers extending radially inward from the distal tip.

14. The system of claim 12, wherein the restriction is clover shaped having three fingers extending radially inward from the distal tip.

15. The system of claim 12, wherein the restriction comprises a plurality of slots.

16. The system of claim 12, wherein the restriction comprises a plurality of aspiration openings.

17. The system of claim 12, wherein the restriction comprises two hemispheric openings.

18. The system of claim 12, wherein the optical sensor is positioned along a first axis parallel to a central axis of the working channel and the distal end of the working channel extension is axially displaced from a distal end of the optical sensor.

19. The system of claim 18, further comprising an ablation device configured to extend through the working channel and the working channel extension, wherein a distal end of the ablation device is configured to be axially aligned with the distal end of the working channel extension.

20. The system of claim 18, wherein distal end of the distal tip is axially displaced from the optical sensor by the second distance.

21. The system of claim 12, wherein the first distance maintains a separation between a distal end of an ablation device and the optical sensor and the second distance maintains a separation between an aspiration opening and the optical sensor.

22. The system of claim 12, wherein the distal tip further comprises a cup, wherein the cup extends distally from the distal end of the working channel extension.

23. A system for removing a biological object from a location internal to a subject, the system comprising:
a supply lumen configured to receive a first fluid flow and to redirect the first fluid flow from a first direction to a second direction;
an evacuation lumen comprising a working channel configured to receive the first fluid flow and a second fluid flow travelling in the second direction from the location internal to the subject toward the evacuation lumen, the second fluid flow being configured to carry at least a portion of the biological object;
an aspiration opening in fluid communication with the evacuation lumen, wherein the aspiration opening is configured to receive the second fluid flow from the location internal to the subject as a result of the first fluid flow entering the evacuation lumen; and
a distal tip extending distally from a distal end of the evacuation lumen, the distal tip comprising a working channel extension extending from a distal end of the distal tip to a proximal end of the distal tip, wherein the working channel extension is in fluid communication with the working channel, wherein the distal tip is configured to enhance visibility for an optical sensor configured to provide images of the location internal to the subject, and wherein the distal tip is further configured to prevent the biological object from clogging the working channel.

24. The system of claim 23, wherein the distal tip comprises a choke positioned in the working channel extension and defining one or more constrictions at one or more axial positions between the distal end and the proximal end of the distal tip, and wherein the choke has an effective cross-sectional area smaller than a cross-sectional area of the evacuation lumen.

25. The system of claim 23, wherein the distal tip further comprises a cup having an annular wall extending distally from the distal end of the evacuation lumen, and wherein the cup is configured to retain the biological object near the aspiration opening.

26. The system of claim 23, wherein the distal tip is formed from a laser energy resistant material.

27. The system of claim 23, wherein the distal tip is formed from an optically transparent material.

28. The system of claim 23, wherein the evacuation lumen and the distal tip are configured to receive an ablation device.

29. The system of claim 28, further comprising a mechanism configured to move the ablation device in the evacuation lumen.
